SEQUENCE LISTING (2 sequences)

SEQ ID NO: 1 - protein, 1593 aa, Homo sapiens
MPCAQRSWLANLSVVAQLLNFGALCYGRQPQPGPVRFPDRRQEHFIKGLPEYHVVGPVRVDASGHFLSYGLHYPITSSRRKRDLDGSEDWVYYRISHEEKDLFFNLTVNQGFLSNSYIMEKRYGNLSHVKMMASSAPLCHLSGTVLQQGTRVGTAALSACHGLTGFFQLPHGDFFIEPVKKHPLVEGGYHPHIVYRRQKVPETKEPTCGLKDSVNISQKQELWREKWERHNLPSRSLSRRSISKERWVETLVVADTKMIEYHGSENVESYILTIMNMVTGLFHNPSIGNAIHIVVVRLILLEEEEQGLKIVHHAEKTLSSFCKWQKSINPKSDLNPVHHDVAVLLTRKDICAGFNRPCETLGLSHLSGMCQPHRSCNINEDSGLPLAFTIAHELGHSFGIQHDGKENDCEPVGRHPYIMSRQLQYDPTPLTWSKCSEEYITRFLDRGWGFCLDDIPKKKGLKSKVIAPGVIYDVHHQCQLQYGPNATFCQEVENVCQTLWCSVKGFCRSKLDAAADGTQCGEKKWCMAGKCITVGKKPESIPGGWGRWSPWSHCSRTCGAGVQSAERLCNNPEPKFGGKYCTGERKRYRLCNVHPCRSEAPTFRQMQCSEFDTVPYKNELYHWFPIFNPAHPCELYCRPIDGQFSEKMLDAVIDGTPCFEGGNSRNVCINGICKMVGCDYEIDSNATEDRCGVCLGDGSSCQTVRKMFKQKEGSGYVDIGLIPKGARDIRVMEIEGAGNFLAIRSEDPEKYYLNGGFIIQWNGNYKLAGTVFQYDRKGDLEKLMATGPTNESVWIQLLFQVTNPGIKYEYTIQKDGLDNDVEQMYFWQYGHWTECSVTCGTGIRRQTAHCIKKGRGMVKATFCDPETQPNGRQKKCHEKACPPRWWAGEWEACSATCGPHGEKKRTVLCIQTMVSDEQALPPTDCQHLLKPKTLLSCNRDILCPSDWTVGNWSECSVSCGGGVRIRSVTCAKNHDEPCDVTRKPNSRALCGLQQCPSSRRVLKPNKGTISNGKNPPTLKPVPPPTSRPRMLTTPTGPESMSTSTPAISSPSPTTASKEGDLGGKQWQDSSTQPELSSRYLISTGSTSQPILTSQSLSIQPSEENVSSSDTGPTSEGGLVATTTSGSGLSSSRNPITWPVTPFYNTLTKGPEMEIHSGSGEEREQPEDKDESNPVIWTKIRVPGNDAPVESTEMPLAPPLTPDLSRESWWPPFSTVMEGLLPSQRPTTSETGTPRVEGMVTEKPANTLLPLGGDHQPEPSGKTANRNHLKLPNNMNQTKSSEPVLTEEDATSLITEGFLLNASNYKQLTNGHGSAHWIVGNWSECSTTCGLGAYWKRVECTTQMDSDCAAIQRPDPAKRCHLRPCAGWKVGNWSKCSRNCSGGFKIREIQCVDSRDHRNLRPFHCQFLAGIPPPLSMSCNPEPCEAWQVEPWSQCSRSCGGGVQERGVFCPGGLCDWTKRPTSTMSCNEHLCCHWATGNWDLCSTSCGGGFQKRIVQCVPSEGNKTEDQDQCLCDHKPRPPEFKKCNQQACKKSADLLCTKDKLSASFCQTLKAMKKCSVPTVRAECCFSCPQTHITHTQRQRRQRLLQKSKEL

SEQ ID NO: 2 - DNA, 5070 bp, Homo sapiens
cgctttatttctggagctgagggctaaaacttttttcacttttcttctcctcaacatctgaatcatgccatgtgcccagaggagctggcttgcaaacctttccgtggtggctcagctccttaactttggggcgctttgctatgggagacagcctcagccaggcccggttcgcttcccggacaggaggcaagagcattttatcaagggcctgccagaataccacgtggtgggtccagtccgagtagatgccagtgggcattttttgtcatatggcttgcactatcccatcacgagcagcaggaggaagagagatttggatggctcagaggactgggtgtactacagaatttctcacgaggagaaggacctgttttttaacttgacggtcaatcaaggatttctttccaatagctacatcatggagaagagatatgggaacctctcccatgttaagatgatggcttcctctgcccccctctgccatctcagtggcacggttctacagcagggcaccagagttgggacggcagccctcagtgcctgccatggactgactggatttttccaactaccacatggagactttttcattgaacccgtgaagaagcatccactggttgagggagggtaccacccgcacatcgtttacaggaggcagaaagttccagaaaccaaggagccaacctgtggattaaaggacagtgttaacatctcccagaagcaagagctatggcgggagaagtgggagaggcacaacttgccaagcagaagcctctctcggcgttccatcagcaaggagagatgggtggagacactggtggtggccgacacaaagatgattgaataccatgggagtgagaatgtggagtcctacatcctcaccatcatgaacatggtcactgggttgttccataacccaagcattggcaatgcaattcacattgttgtggttcggctcattctactcgaagaagaagagcaaggactgaaaatagttcaccatgcagaaaagacactgtctagcttctgcaagtggcagaagagtatcaatcccaagagtgacctcaatcctgttcatcacgacgtggctgtccttctcaccagaaaggacatctgtgctggtttcaatcgcccctgcgagaccctgggcctgtctcacctttcaggaatgtgtcagcctcaccgcagttgtaacatcaatgaagattcgggactccctctggctttcacaattgcccatgagctaggacacagcttcggcatccagcatgatgggaaagaaaatgactgtgagcctgtgggcagacatccgtacatcatgtcccgccagctccagtacgatcccactccgctgacatggtccaagtgcagcgaggagtacatcacccgcttcttggaccgaggctgggggttctgtcttgatgacatacctaaaaagaaaggcttgaagtccaaggtcattgcccccggagtgatctatgatgttcaccaccagtgccagctacaatatggacccaatgctaccttctgccaggaagtagaaaacgtctgccagacactgtggtgctccgtgaagggcttttgtcgctctaagctggacgctgctgcagatggaactcaatgtggtgagaagaagtggtgtatggcaggcaagtgcatcacagtggggaagaaaccagagagcattcctggaggctggggccgctggtcaccctggtcccactgttccaggacctgtggggctggagtccagagcgcagagaggctctgcaacaaccccgagccaaagtttggagggaaatattgcactggagaaagaaaacgctatcgcttgtgcaacgtccacccctgtcgctcagaggcaccaacatttcggcagatgcagtgcagtgaatttgacactgttccctacaagaatgaactctaccactggtttcccatttttaacccagcacatccttgtgagctctactgccgacccatagatggccagttttctgagaaaatgctggatgctgtcattgatggtaccccttgctttgaaggcggcaacagcagaaatgtctgtattaatggcatatgtaagatggttggctgtgactatgagatcgattccaatgccaccgaggatcgctgcggtgtgtgcctgggagatggctcttcctgccagactgtgagaaagatgtttaagcagaaggaaggatctggttatgttgacattgggctcattccaaaaggagcaagggacataagagtgatggaaattgagggagctggaaacttcctggccatcaggagtgaagatcctgaaaaatattacctgaatggagggtttattatccagtggaacgggaactataagctggcagggactgtctttcagtatgacaggaaaggagacctggaaaagctgatggccacaggtcccaccaatgagtctgtgtggatccagcttctattccaggtgactaaccctggcatcaagtatgagtacacaatccagaaagatggccttgacaatgatgttgagcagatgtacttctggcagtacggccactggacagagtgcagtgtgacctgcgggacaggtatccgccgccaaactgcccattgcataaagaagggccgcgggatggtgaaagctacattctgtgacccagaaacacagcccaatgggagacagaagaagtgccatgaaaaggcttgtccacccaggtggtgggcaggggagtgggaagcatgctcggcgacatgcgggccccacggggagaagaagcgaaccgtgctgtgcatccagaccatggtctctgacgagcaggctctcccgcccacagactgccagcacctgctgaagcccaagaccctcctttcctgcaacagagacatcctgtgcccctcggactggacagtgggcaactggagtgagtgttctgtttcctgtggtggtggagtgcggattcgcagtgtcacatgtgccaagaaccatgatgaaccttgcgatgtgacaaggaaacccaacagccgagctctgtgtggcctccagcaatgcccttctagccggagagttctgaaaccaaacaaaggcactatttccaatggaaaaaacccaccaacactaaagcccgtccctccacctacatccaggcccagaatgctgaccacacccacagggcctgagtctatgagcacaagcactccagcaatcagcagccctagtcctaccacagcctccaaagaaggagacctgggtgggaaacagtggcaagatagctcaacccaacctgagctgagctctcgctatctcatttccactggaagcacttcccagcccatcctcacttcccaatccttgagcattcagccaagtgaggaaaatgtttccagttcagatactggtcctacctcggagggaggccttgtagctacaacaacaagtggttctggcttgtcatcttcccgcaaccctatcacttggcctgtgactccattttacaataccttgaccaaaggtccagaaatggagattcacagtggctcaggggaagaaagagaacagcctgaggacaaagatgaaagcaatcctgtaatatggaccaagatcagagtacctggaaatgacgctccagtggaaagtacagaaatgccacttgcacctccactaacaccagatctcagcagggagtcctggtggccacccttcagcacagtaatggaaggactgctccccagccaaaggcccactacttccgaaactgggacacccagagttgaggggatggttactgaaaagccagccaacactctgctccctctgggaggagaccaccagccagaaccctcaggaaagacggcaaaccgtaaccacctgaaacttccaaacaacatgaaccaaacaaaaagttctgaaccagtcctgactgaggaggatgcaacaagtctgattactgagggctttttgctaaatgcctccaattacaagcagctcacaaacggccacggctctgcacactggatcgtcggaaactggagcgagtgctccaccacatgtggcctgggggcctactggaaaagggtggagtgcaccacccagatggattctgactgtgcggccatccagagacctgaccctgcaaaaagatgccacctccgtccctgtgctggctggaaagtgggaaactggagcaagtgctccagaaactgcagtgggggcttcaagatacgcgagattcagtgcgtggacagccgggaccaccggaacctgaggccatttcactgccagttcctggccggcattcctcccccattgagcatgagctgtaacccggagccctgtgaggcgtggcaggtggagccttggagccagtgctccaggtcctgtggaggtggagttcaggagagaggagtgttctgtccaggaggcctctgtgattggacaaaaagacccacatccaccatgtcttgcaatgagcacctgtgctgtcactgggccactgggaactgggacctgtgttccacttcctgtggaggtggctttcagaagaggattgtccaatgtgtgccctcagagggcaataaaactgaagaccaagaccaatgtctatgtgatcacaaacccagacctccagaattcaaaaaatgcaaccagcaggcctgcaagaaaagtgccgatttactttgcactaaggacaaactgtcagccagtttctgccagacactgaaagccatgaagaaatgttctgtgcccaccgtgagggctgagtgctgcttctcgtgtccccagacacacatcacacacacccaaaggcaaagaaggcaacggttgctccaaaagtcaaaagaactctaagcccaaaaaggaagccagcttccaccagactgcagcagcctgctgaccatgacatgctttagccctgggagctgcttcgttgaggttttgcttcatatatttcagattcagctttgggtcatgatgaaacaaaatccattgtgttcctctagccaccgaatgtccctcacaggaggcctaggctgccgtacttgctgctccctgataggaaggtgaaatagcta

(19) United States
(12) Patent Application Publication
(10) Pub. No.: US 2004/0224378 A1
(43) Pub. Date: Nov. 11, 2004

(54) Methods for using ADAMTS-12, an integrin and metalloprotease with thrombspondin motifs

(75) Inventors: Paul David Jackson, Shaker Heights, OH (US); Anthony Eugene Ting, Shaker Heights, OH (US); Pieter W. Faber, Westlake, OH (US)

Correspondence Address: LAHIVE & COCKFIELD, LLP., 28 STATE STREET, BOSTON, MA, 02109, US

(73) Assignee: Athersys, Inc., Cleveland, OH

(21) Appl. No.: 10/164,893

(22) Filed: Jun. 7, 2002

(51) Int. Cl. C12Q 1/37

(52) U.S. Cl. 435/23

(57) ABSTRACT

The present invention relates to methods for using a human A Disintegrin And Metalloprotease containing ThromboSpondin repeats (ADAM-TS). The invention also relates to methods for using polynucleotides encoding the metalloprotease. The invention relates to methods using the ADAM-TS polypeptides and polynucleotides as a target for diagnosis and treatment in metalloprotease-mediated or -related disorders. The invention further relates to drug-screening methods using the ADAM-TS polypeptides and polynucleotides to identify agonists and antagonists for diagnosis and treatment. The invention further encompasses agonists and antagonists based on the ADAM-TS polypeptides and polynucleotides. The invention further relates to agonists and antagonists identified by drug screening methods with the ADAM-TS polypeptides and polynucleotides as a target. The invention further related to methods of treating a subject suffering from a ADAM-TS associated disorder.

### BACKGROUND OF THE INVENTION

Metalloproteases are a ubiquitous class of enzymes that have been implicated in a large number of cellular processes. Despite the fact that these enzymes differ widely in their sequence and structure they all rely on the action of a catalytically active metal atom. In most instances this metal is a zinc atom, but examples are known which contain cobalt or nickel. Metalloproteases use the catalytic metal atom to catalyze hydrolysis of peptide bonds (for a review, see Power and Harper, in Protease Inhibitors, A. J. Barrett and G. Salversen (eds.) Elsvier, Amsterdam, 1986, p. 219). Currently, metalloproteases have been divided into at least five distinct families based on structure and activity.

The members of one family of metalloproteases known as ADAM (A Disintegrin And Metalloprotease) display the common characteristic of being type I transmembrane gylcoproteins that are important in diverse biologic processes, such as cell adhesion and proteolytic shedding of cell surface receptors. Structurally, ADAMs consist of a prodomain that blocks protease activity; a zinc-binding metalloprotease domain; disintegrin and cysteine-rich domains with adhesion activity; an epidermal growth factor-like domain with cell fusion activity; a transmembrane domain; and a phosphorylated cytoplasmic regulatory domain. These enzymes have been implicated in a variety of cell-cell and cell-matrix interactions (Werb (1997) Cell 91:439-442; Blobel, (1997) Cell 91:275-278).

A subfamily of ADAMs can be distinguished from other ADAM family members based on the multiple copies of thrombospondin 1-like repeats they carry. With at least ten members in mammals alone, the ADAM-TS (a disintegrin and metalloprotease with thromobospondin motifs) subfamily members are predicted by their structural domains to be extracellular matrix (ECM) proteins with a wide range of activities and functions distinct from members of the ADAM family. The first member of this subfamily, ADAM-TS1, was identified as a consequence of its association with the development of cancer cachexia as well as with various inflammatory processes (Kuno, et al., (1997) J. Biol. Chem. 272:556-562). Procollagen I amino-proteinase, ADAM-TS2, is associated with Ehlers-Danlos syndrome type VIIC in humans (Colige, et al. (1999) Am. J. Hum. Genet. 65:308-317). ADAM-TS4 and ADAM-TS5/TS11 have been characterized as aggrecanases responsible for the degradation of cartilage aggrecan in arthritic diseases (Tortorella et al. (1999) Science 284;1664-1666; Abbaszade et al. (1999) J. Biol. Chem. 274:23443-23450). ADAM-TS4 has been found to be responsible for brevican cleavage in glioma cells, a proteolytic cleavage that has been proposed to be critical in mediating the invasiveness of these tumors (Matthews, et al. (2000) J. Biol. Chem. 275:22695-22703). ADAM-TS8 (also called METH-2) and ADAM-TS1 have been characterized as proteins with angio-inhibitory activity (Vasquez et al. (1999) J. Biol. Chem. 274:23349-23357). Other members of this family such as ADAM-TS3, ADAM-TS5, ADAM-TS6, ADAM-TS7, and ADAM-TS9 have been only characterized at the structural level and their putative functional significance is still unclear.

In view of the importance of cell-cell and cell-matrix interactions in both normal and pathological conditions, there is a ongoing need for the identification and biological characterization of additional members of the ADAM family of metalloproteases in order to elucidate those members which are valuable drug targets, as well as prognostic and diagnostic markers for a variety of pathological processes.

### SUMMARY OF THE INVENTION

The present invention is based on the discovery that ADAMTS-12, a recently identified member of the ADAM-TS subfamily of metalloproteases, is a key element effecting changes in extracellular matrix and cell motility. Accordingly, alterations in ADAMTS-12 expression or activity play a key role in disorders related to aberrant changes in extracellular matrix formation or breakdown, and/or decrease or increase in cell motility. For example, disorders in which extracellular matrix breakdown and increased cell motility have been observed, include, but are not limited to, neoplastic conditions such as tumor formation, invasiveness and metastasis; inflammatory conditions such as arthritis (e.g., rheumatoid arthritis and osteoarthritis); and cardiovascular conditions including atherosclerosis, restenosis, dilated cardiomyopathy, cardiac hypertrophy and myocardial infarction.

Accordingly, it is an object of the invention to provide methods wherein ADAMTS-12 polypeptides are useful as reagents or targets in metalloprotease, extracellular matrix and/or cell motility assays that are applicable to treatment and diagnosis of diseases or disorders related to extracellular matrix breakdown and/or increased cell motility mediated by, or related to, ADAMTS-12.

It is a further object of the invention to provide methods wherein polynucleotides corresponding to the ADAMTS-12 polypeptides are useful as probes, targets or reagents that are applicable to treatment and diagnosis of diseases or disorders related to extracellular matrix breakdown and/or increased cell motility mediated by, or related to, ADAMTS-12.

A specific object of the invention is to identify compounds that act as agonists and antagonists and modulate the expression of ADAMTS-12 in cells or tissues. Such compounds can be used to alter extracellular matrix formation or breakdown, and/or cell motility in diseases mediated by, or related to, ADAMTS-12. Accordingly, in one aspect the invention provides methods of screening for compounds that modulate expression or activity of the ADAMTS-12 polypeptides or nucleic acids (RNA or DNA) in cells or tissues. In certain embodiments, the cells or tissues are derived from cells or tissues in which ADAMTS-12 expression or activity has been altered, e.g., from animals or individuals having a disorder mediated by or related to ADAMTS-12.

A further object of the invention is to provide compounds that modulate expression of ADAMTS-12 for treatment and diagnosis of disorders mediated by or related to ADAMTS-12, such as neoplastic conditions such as tumor formation, invasiveness and metastasis; inflammatory conditions such as arthritis (e.g., rheumatoid arthritis and osteoarthritis); and cardiovascular conditions including atherosclerosis, restenosis, dilated cardiomyopathy, cardiac hypertrophy and myocardial infarction.

The invention also provides a process for modulating ADAMTS-12 polypeptides or nucleic acid expression or activity, especially using the screened compounds. Modulation can be used to treat conditions related to aberrant activity or expression of the ADAMTS-12 polypeptides or nucleic acids.

The invention further provides assays for determining the activity of, or the presence or absence of ADAMTS-12 polypeptides or nucleic acid molecules in biological samples, including for disease diagnosis.

The invention also provides assays for determining the presence of a mutation in ADAMTS-12 polypeptides or nucleic acid molecules, including for disease diagnosis.

The invention utilizes isolated ADAMTS-12 polypeptides, including a polypeptide having the amino acid sequence shown in SEQ ID NO 1.

The invention also utilizes isolated ADAMTS-12 nucleic acid molecule having the sequence shown in SEQ ID NO 2 or a complement thereof.

The invention also utilizes variant polypeptides having an amino acid sequence that is substantially homologous to the amino acid sequence shown in SEQ ID NO 1.

The invention also utilizes variant nucleic acid sequences that are substantially homologous to the nucleotide sequence shown in SEQ ID NO 2.

The invention also utilizes fragments of the polypeptide shown in SEQ ID NO 1 and nucleotide sequence shown in SEQ ID NO 2, complements of the nucleotide sequence shown in SEQ ID NO 2, as well as substantially homologous fragments of the polypeptide or nucleic acid.

The invention further utilizes nucleic acid constructs comprising the nucleic acid molecules described herein. In certain embodiments, the nucleic acid molecules of the invention are operatively linked to a regulatory sequence.

The invention also utilizes vectors and host cells that express ADAMTS-12 and provides methods for expressing ADAMTS-12 nucleic acid molecules and polypeptides in cells, and particularly recombinant vectors and host cells.

The invention also utilizes methods of making the vectors and host cells and provides methods for using them to assay expression and cellular effects of expression of the ADAMTS-12 nucleic acid molecules and polypeptides in specific cell types and disorders.

The invention also utilizes antibodies or antigen-binding fragments thereof that selectively bind the ADAMTS-12 polypeptides and fragments.

### DESCRIPTION OF THE DRAWINGS

FIG. 1 shows the nucleotide (SEQ ID NO:2) and the deduced amino acid sequence (SEQ ID NO:1) of ADAMTS-12.

FIG. 2 shows lists the Accession Numbers for the amino acid sequences of the various ADAM-TS subfamily members, and depicts the amino acid alignment of these ADAM-TS polypeptides. Regions of consensus are depicted on the top line.

FIG. 3 depicts the various domains of the ADAMTS-12 polypeptide FIG. 4 depicts an analysis of the ADAMTS-12 amino acid sequence: alpha, beta, turn and coil regions; hydrophilicity plot; hydrophobicity plot; alpha and beta amphipathic regions; flexable regions; antigenic index; and surface probability plot.

FIG. 5 depicts the phylogenetic relationship between members of the ADAM-TS subfamily.

### DETAILED DESCRIPTION OF THE INVENTION

The increased number of cancer cases reported in the United States and around the world is a major concern. Progress in finding effective treatments for various types of cancer is ongoing, but the most effective treatments currently in clinical use require not only an early detection, but also close monitoring of patients after treatment to prevent tumor recurrence. Further, a reliable assessment of malignancy of many cancer types is needed due to the fact that the spread of cancer cells from a primary tumor to distant organs is the major cause of death in cancer patients. This is because metastatic lesions are often resistant to traditional cancer therapies due to the progressive phenotyptic changes that the cancer cells have undergone. Therefore methods for determining whether an individual has developed, or is likely to develop, metastatic cancer would aid the clinician in choosing the most efficacious therapy regimen.

Other progressive diseases in which reliable treatments have remained elusive include inflammatory diseases such as arthritis (e.g., osteoarthritis, rheumatoid arthritis), and cardiovascular diseases including atherosclerosis, restenosis, dilated cardiomyopathy, myocardial infarction and cardiac hypertrophy. Therefore, methods for treating the developing pathology of these diseases would also be highly beneficial.

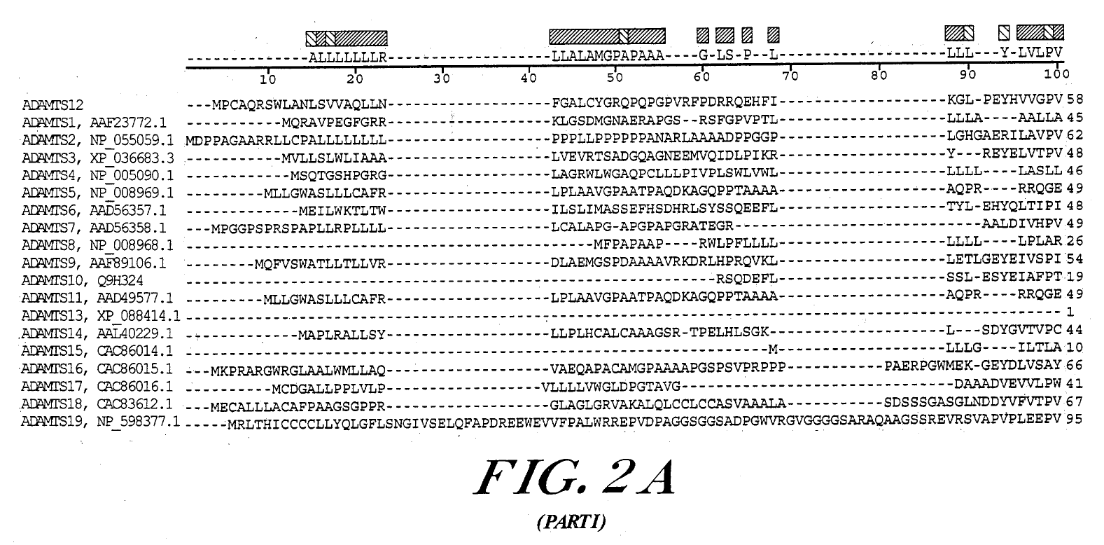

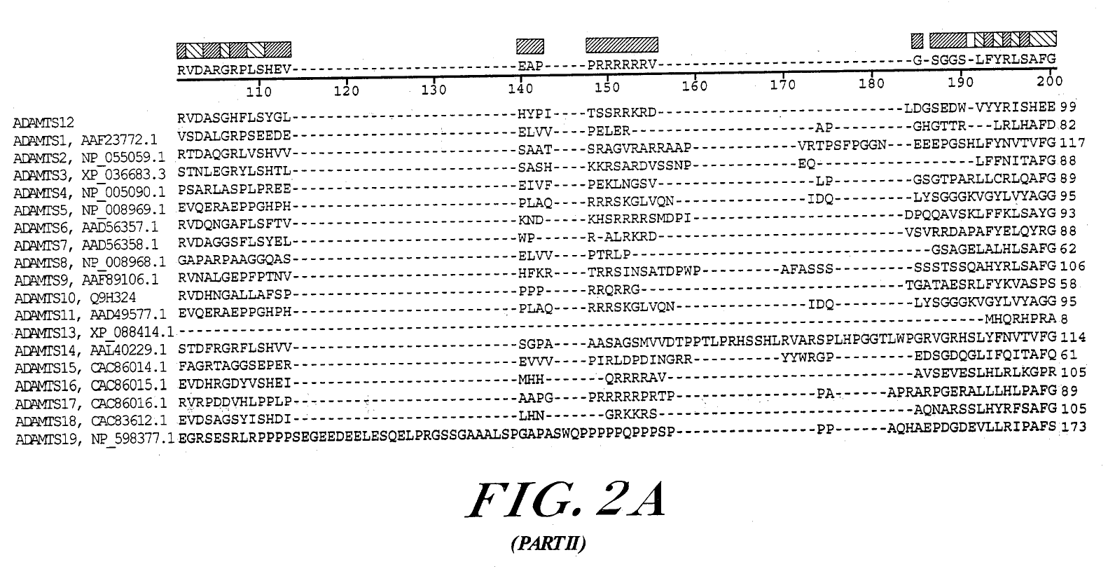

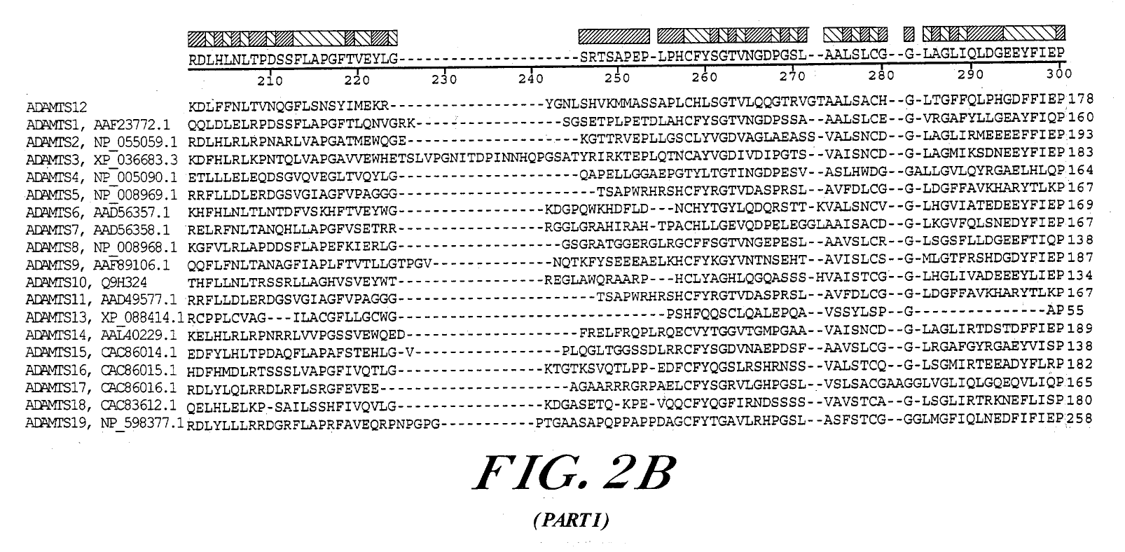

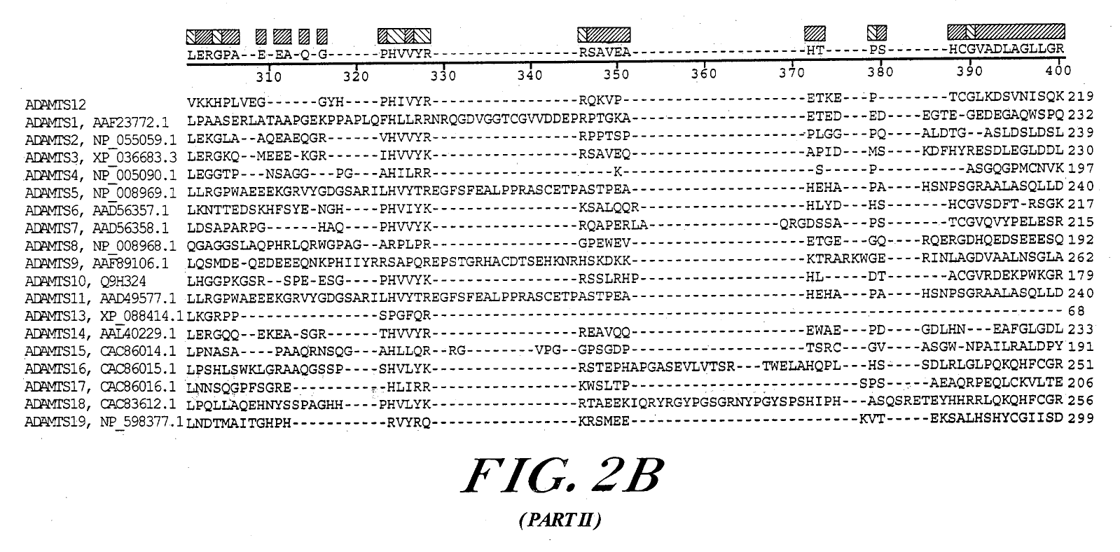

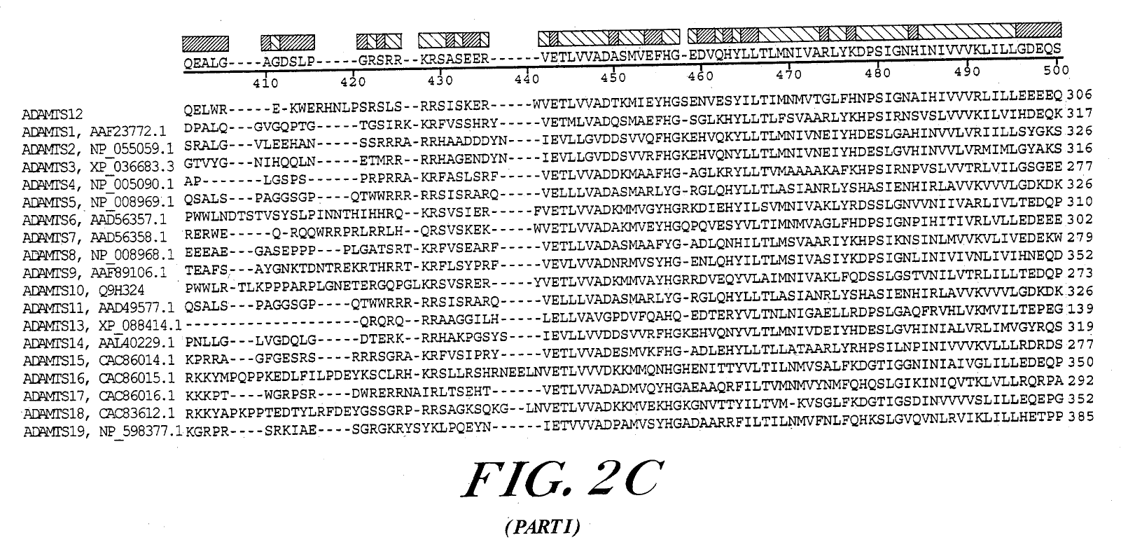

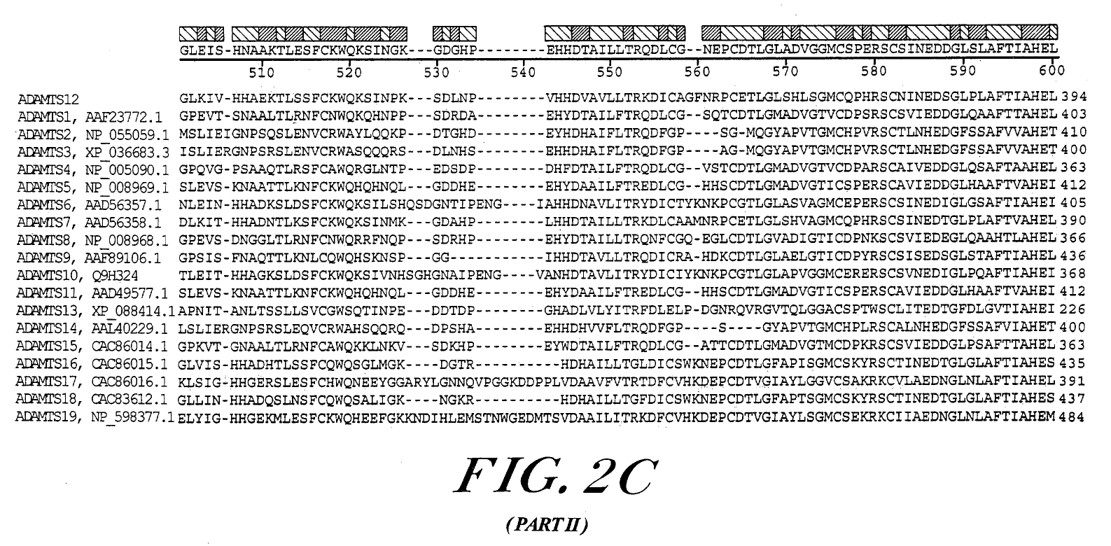

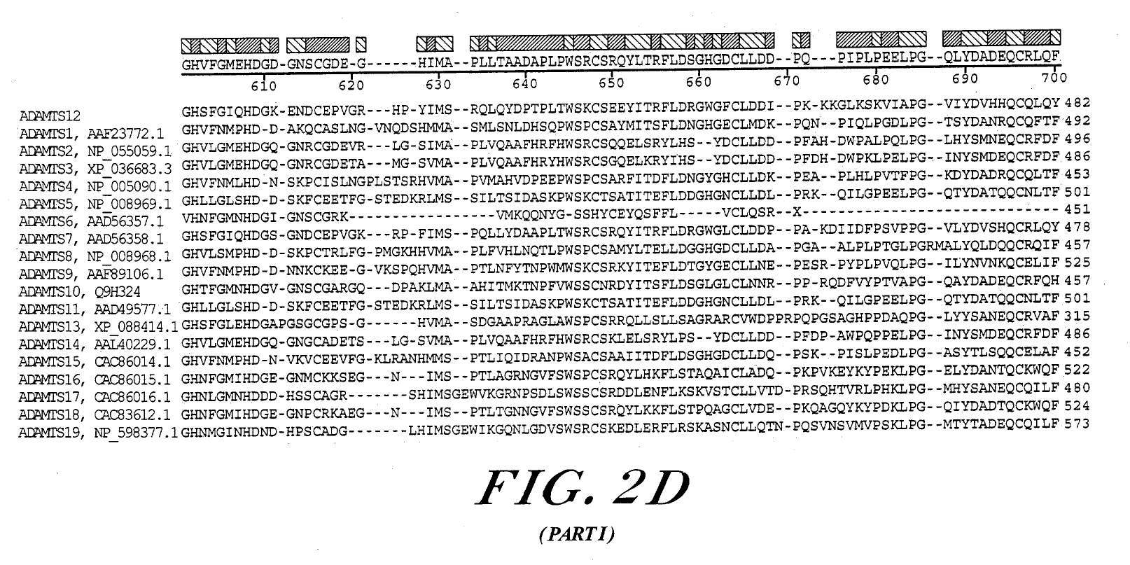

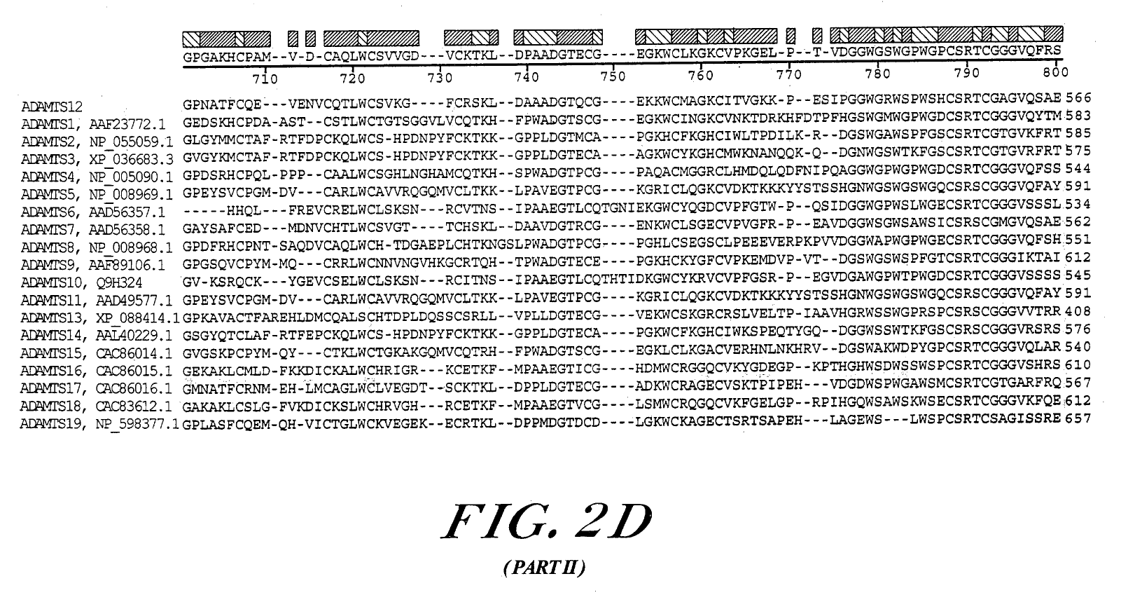

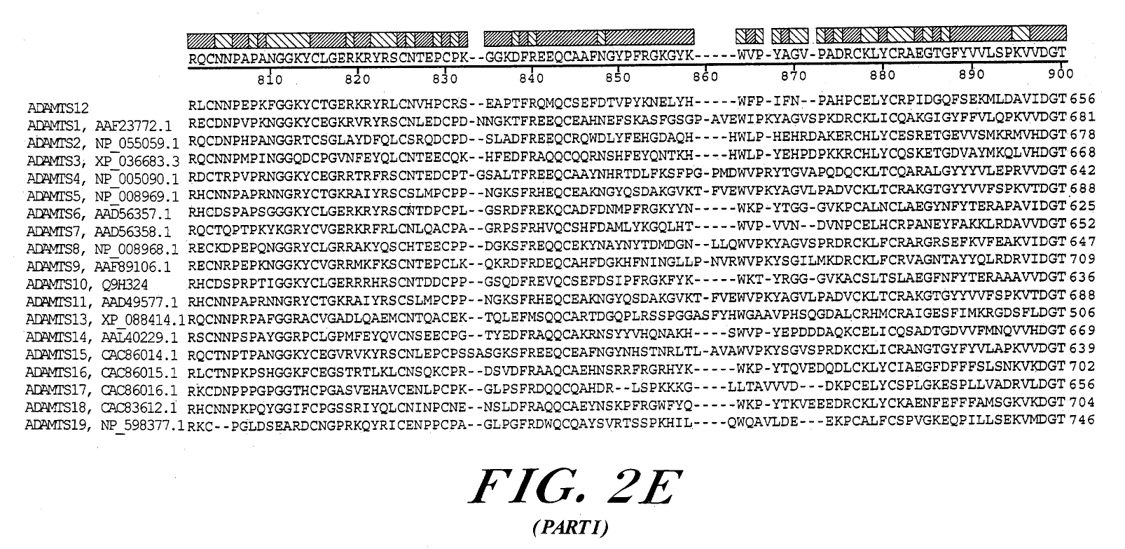

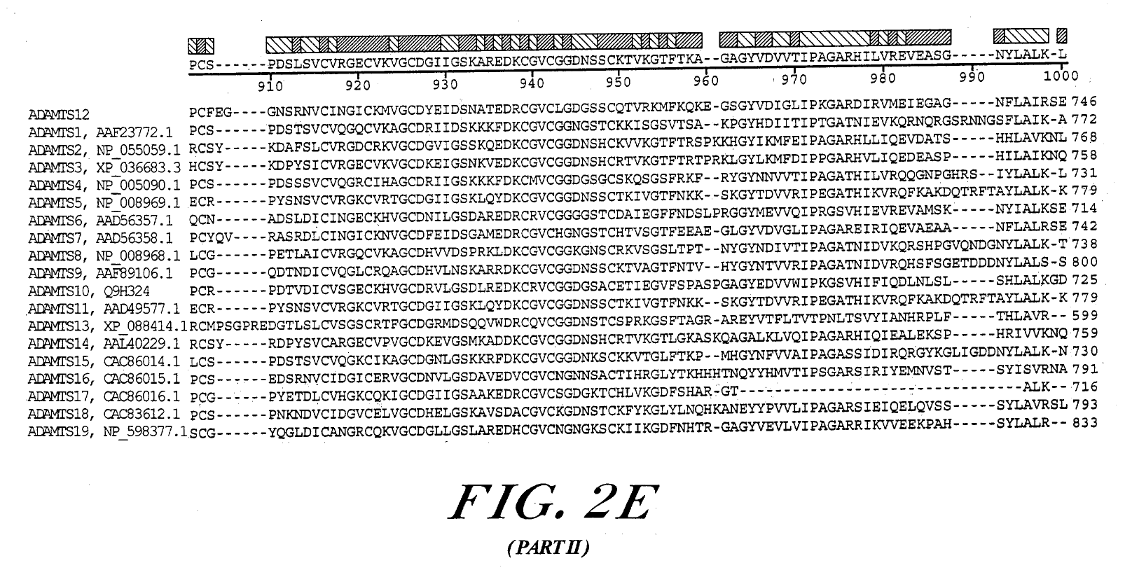

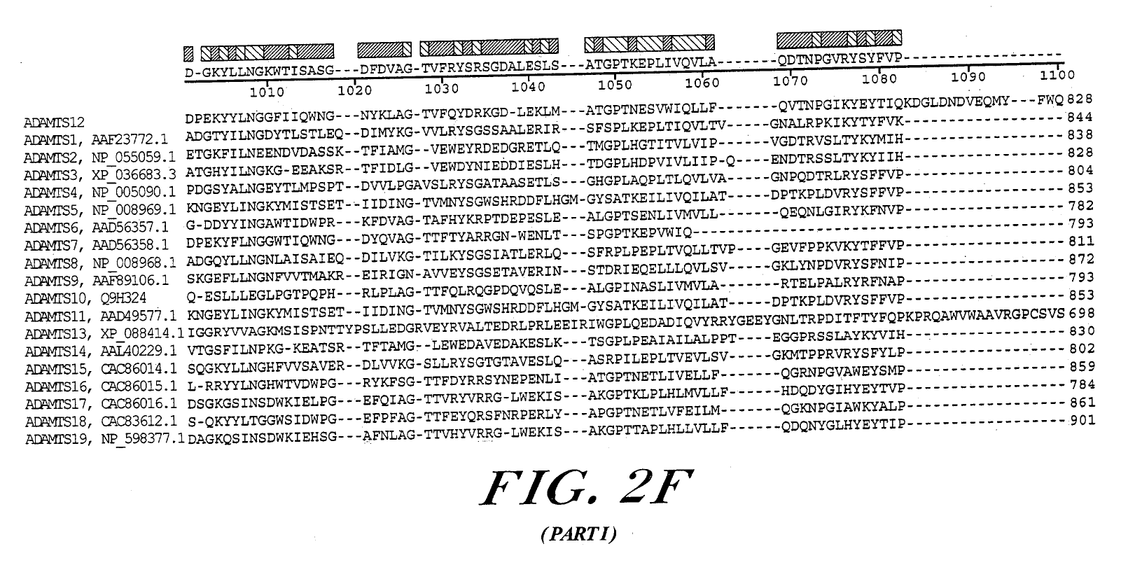

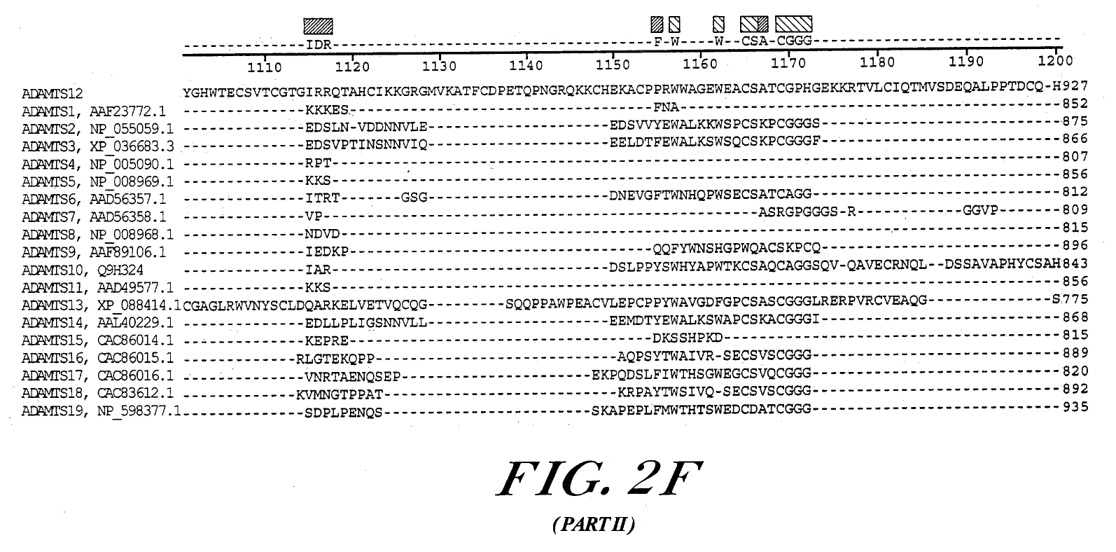

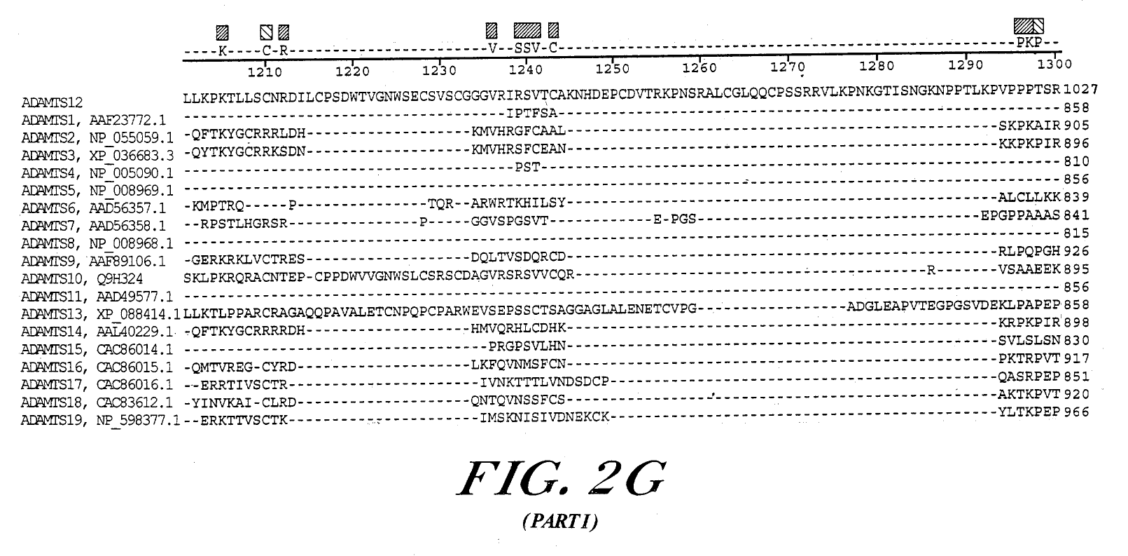

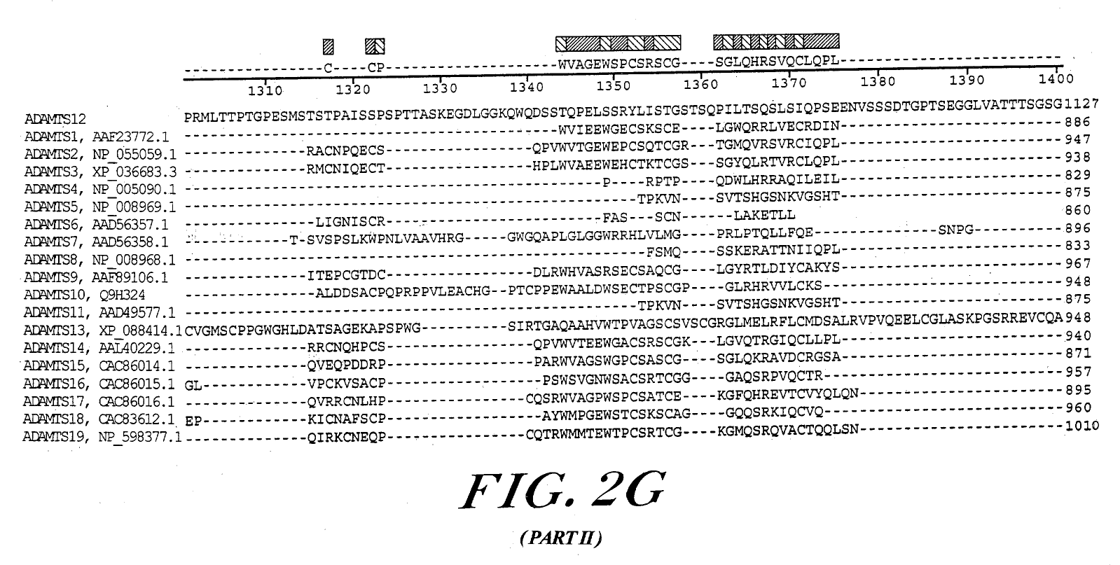

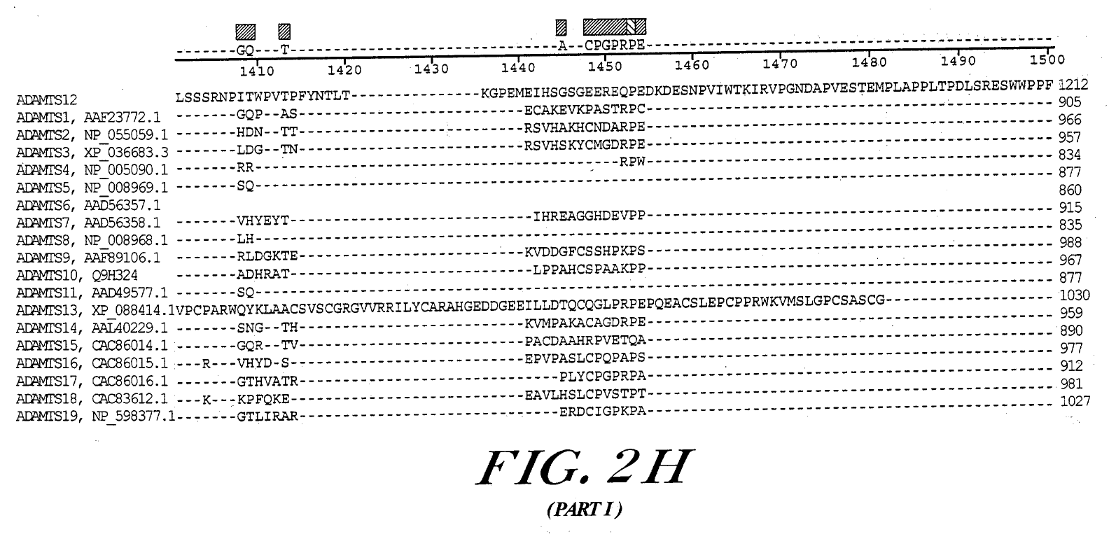

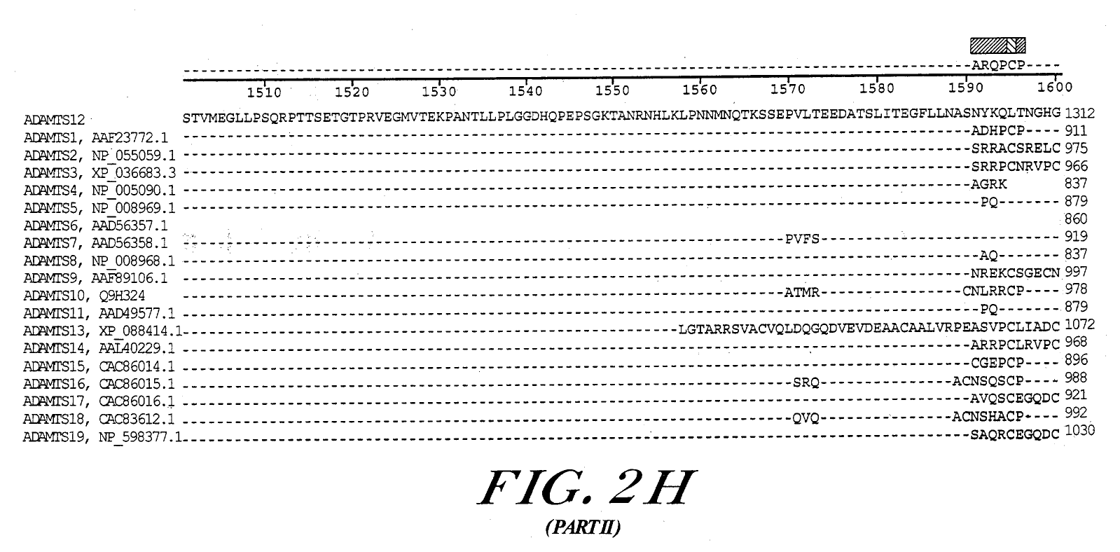

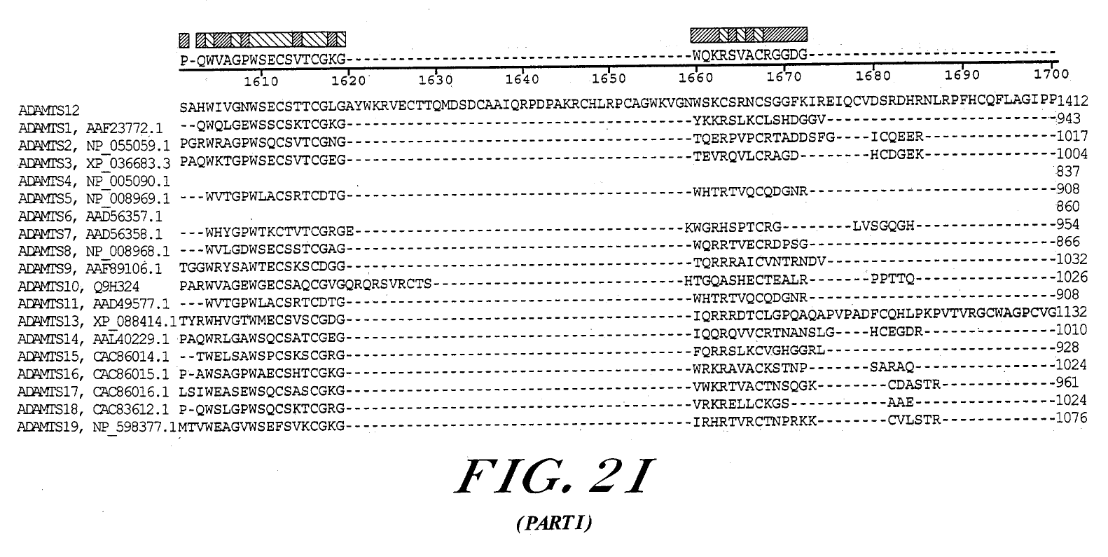

Applicants have discovered that aberrant expression of the metalloprotease, ADAMTS-12, results in increased breakdown of the extracellular matrix and increased cell motility, as well as tumor invasiveness and metastasis. This metalloprotease exhibits a similar domain organization to the other members of the ADAM-TS subfamily, with the exception that ADAMTS-12 contains a unique number and organization of TS repeats. This protein also contains thrombospondin repeats which are organized into three groups. An alignment of the amino acid sequences of various ADAM-TS polypeptides with ADAMTS-12 is depicted in FIG. 2, and the structure of the ADAMTS-12 protein is depicted in FIG. 3. Overexpression of ADAMTS-12 in diseases and disorders associated with breakdown of the extracellular matrix, indicates, for the first time, that this molecule is an important drug target, and that assays that utilize ADAMTS-12 provide a novel approach to identifying those individuals who have, or are at risk of developing ADAMTS-12 disorders. For example, the methods of the invention are especially useful for identifying individuals whose tumors have metastasized, or have tumors that are likely to metastasize.

Accordingly, in one aspect, the invention provides methods and reagents for diagnosing diseases and disorders associated with the breakdown of the extracellular matrix, and/or increased cell motility mediated by ADAMTS-12, methods for measuring the aggressiveness of diseases and disorders associated with the breakdown of the extracellular matrix and/or increased cell motility mediated by ADAMTS-12. For example, in certain embodiments, the invention includes methods for identifying cancers that have metastasized or are likely to metastasize. The diagnostic and prognostic assays of this invention include methods involving antibody-based detection of ADAMTS-12 polypeptides, and nucleic acid-based detection of ADAMTS-12 mRNA and DNA.

In one embodiment, this invention provides a method for identifying a disorder related to increased extracellular matrix breakdown in a subject, comprising: assessing the level of ADAMTS-12 in a biological sample from the subject, wherein an elevation in the level of ADAMTS-12 is indicative of a disorder related to increased extracellular matrix breakdown.

In another embodiment, this invention provides a method for identifying a disorder related to increased cell motility in a subject, comprising: assessing the level of ADAMTS-12 in a biological sample from the subject, wherein an elevation in the level of ADAMTS-12 is indicative of a disorder related to increased cell motility.

In a related embodiment this invention provides a method for identifying metastatic cancer in a subject, comprising: assessing the level of ADAMTS-12 in a biological sample from the subject, wherein an elevation in the level of ADAMTS-12 is indicative of metastatic cancer.

In a further related embodiment, this invention provides a method for identifying metastatic cancer in a subject, comprising: assessing the level of ADAMTS-12 in a biological sample from the subject, wherein an elevation in the level of ADAMTS-12 is indicative of metastatic cancer, and wherein the subject was previously identified as having cancer.

In a related aspect, this invention includes a method for measuring the aggressiveness of cancer in a subject, comprising assessing the level of ADAMTS-12 in a biological sample from the subject, wherein an elevation in the level of ADAMTS-12 is indicative of the aggressiveness of the cancer.

The invention further provides a method for identifying a subject at risk for developing a disorder involving extracellular matrix breakdown and/or increased cell motility comprising: assessing the level of ADAMTS-12 in a biological sample from the subject, wherein an elevation in the level of ADAMTS-12 is indicative that the subject is at risk for developing a disorder involving extracellular matrix breakdown.

The invention further provides a method for identifying a subject at risk for developing metastatic cancer comprising assessing the level of ADAM-TS in a biological sample from the subject, wherein an elevation in the level of ADAMTS-12 is indicative that the subject is at risk for developing metastatic cancer.

Further, provided by this invention are the above methods, wherein assessing the level of ADAMTS-12 in a biological sample from the subject includes contacting the biological sample with an antibody to ADAMTS-12 or a fragment thereof; determining the amount of binding of the antibody to the biological sample; and comparing the amount of antibody bound to the biological sample to a predetermined base level. The amount of binding of the antibody to the biological sample can be determined by the intensity of the signal emitted by the labeled antibody and/or by the number cells in the biological sample bound to the labeled antibody.

Also encompassed by this invention are the above methods wherein the level of ADAMTS-12 is assessed by detecting a level of ADAMTS-12 nucleic acid in a biological sample; and comparing the level of ADAMTS-12 in the biological sample with a level of ADAMTS-12 in a control sample. For example, in certain embodiments ADAMTS-12 nucleic acid is detected using hybridization probes and/or nucleic acid amplification methods.

The diagnostic and prognostic assays of this invention can be further be used in combination with other methods of disease diagnosis. Examples of diagnostic methods that can be used in combination with the assays of the invention include, but are not limited to, current diagnostic methods known to medical practitioners skilled in the art such as ultrasonography or magnetic resonance imaging (MRI), bone scanning, X-rays, skeletal survey, intravenous pyelography, CAT-scan, and biopsy.

The diagnostic and prognostic assays of this invention are particularly useful when the other methods of identifying disorders related to extracellular matrix breakdown and/or changes in cell motility lead to an ambiguous diagnosis. For example, in certain embodiments, the diagnostic and prognostic assays are particularly useful for identifying cancer, particularly metastatic cancer, when the results of other methods fall within a gray zone where it is not clear whether aggressive forms of treatment should be used.

In related embodiments, the diagnostic and prognostic assays of this invention can also be used in combination with other methods of disease staging. The assays of this invention are particularly useful when conventional staging methods leave unclear the prognosis of the disease. For example, in particular embodiments the assays are useful for determining the likelihood of metastatic disease.

The present invention also includes methods of determining whether a subject is likely to respond to a treatment regimen comprising agents, or modulators which have a stimulatory or inhibitory effect on ADAMTS-12 activity (e.g., ADAMTS-12 gene expression or enzyme activity). For example, ADAMTS-12 inhibitors can be administered to individuals, such as those identified using the diagnostic and prognostic methods of the invention as having elevated levels of ADAMTS-12, to treat (prophylactically or therapeutically) disorders (e.g, neoplastic, inflammatory and cardiovasular disorders) associated with aberrant ADAMTS-12 activity.

The diagnostic and prognostic assays of this invention are also useful in assessing the recovery of a subject who is receiving, or has received, therapy for a state associated with aberrant ADAMTS-12 expression or activity. For example, the assays of this invention can be used alone or in combination with other diagnostic methods to assess recovery after treatment, and to monitor for disease recurrence.

The invention also provides methods for diagnosing active disease, or predisposition to disease, in a patient having a variant metalloprotease. Thus, ADAMTS-12 can be isolated from a biological sample and assayed for the presence of a genetic mutation that results in an aberrant protein. This includes amino acid substitution, deletion, insertion, rearrangement, (as the result of aberrant splicing events), and inappropriate post-translational modification. Analytic methods include altered electrophoretic mobility, altered tryptic peptide digest, altered metalloprotease activity in cell-based or cell-free assay, alteration in substrate binding or hydrolysis, or antibody-binding pattern, altered isoelectric point, direct amino acid sequencing, and any other of the known assay techniques useful for detecting mutations in a protein in general or in a metalloprotease specifically. Mutations resulting in aberrant levls of ADAMTS-12 expression can be further identified using standard nucleic acid detection techniques such as those described herein. Mutations resulting in aberrant ADAMTS-12 protein activity can be further identified by assays measuring the extracellular matrix, cell motility or metalloprotease activity, such as, but not limited to those described herein.

The invention also encompasses kits for detecting the presence of a ADAMTS-12 polypeptide or nucleic acid in a biological sample according to the methods described herein. Such kits can be used to determine if a subject is suffering from or is at increased risk of developing a disorder related to extracellular matrix breakdown and/or cell motility (e.g., neoplastic, inflammatory, and cardiovascular disorders), and for identifying subjects who have, or are at risk of developing such disorders. For example, the kit can comprise a labeled compound or agent capable of detecting ADAMTS-12 polypeptide or an mRNA encoding a ADAMTS-12 in a biological sample and means for determining the amount of the ADAMTS-12 polypeptide or ADAMTS-12 mRNA in the sample (e.g., an antibody which binds the polypeptide or an oligonucleotide probe which binds to DNA or mRNA encoding ADAMTS-12). Kits can also include immunomagnetic beads that can be used to facilitate serum assays. Kits can further include instructions for carrying out the methods of the invention and/or for interpreting the results obtained from using the kit.

For example, antibody-based kits can comprise: (I) a first antibody (e.g., attached to a solid support) which binds to a ADAMTS-12 polypeptide; and, optionally, (2) a second, different antibody which binds to either the ADAMTS-12 polypeptide or the first antibody and is conjugated to a detectable label. Nucleic acid-based-kits can comprise, for example: (1) an oligonucleotide, e.g., a detectably labeled oligonucleotide, which hybridizes to a nucleic acid encoding ADAMTS-12 or (2) a pair of primers useful for amplifying a nucleic acid molecule encoding ADAMTS-12. Kits can also comprise a buffering agent, a preservative, or a protein stabilizing agent, components necessary for detecting the detectable label (e.g., an enzyme or a substrate), a control sample or a series of control samples which can be assayed and compared to the biological sample. Each component of the kit can also be enclosed within an individual container, and all of the various containers can be within a single package, along with instructions for interpreting the results of the assays performed using the kit.

In another aspect the invention provides methods for identifying modulators of ADAMTS-12 protein activity or ADAMTS-12 gene expression. These modulators can be used in the treatment of ADAMTS-12 related conditions such as those described herein.

Accordingly, in certain embodiments, the invention provides methods for identifying agents that interact with the ADAMTS-12 protein. This interaction can be detected by functional assays, such as assays for metalloprotease activity, extracellular matrix breakdown or cell motility. Determining the ability of the test compound to interact with ADAMTS-12 can also comprise determining the ability of the test compound to preferentially bind to the polypeptide as compared to the ability of a known binding molecule to bind the polypeptide.

In related embodiments, the invention provides methods to identify agents that modulate metalloprotease activity. Such agents, for example, can increase or decrease affinity or rate of binding to substrate for binding to the ADAMTS-12, or displace the substrate bound to the metalloprotease. For example, both ADAMTS-12 and appropriate variants and fragments can be used in high-throughput screens to assay candidate compounds for the ability to bind to the metalloprotease. These compounds can be further screened against a functional ADAMTS-12 to determine the effects of the compound on the metalloprotease activity, extracellular matrix breakdown or cell motility. Compounds can be identified that activate (agonist) or inactivate (antagonist) the metalloprotease to a desired degree. Modulatory methods can be performed in vitro (e.g., by culturing the cell with the agent) or, alternatively, in vivo (e.g., by administering the agent to a subject). The subject can be a human subject, for example, a subject in a clinical trial or undergoing treatment or diagnosis, or a non-human transgenic subject, such as a transgenic animal model for disease.

Accordingly, the invention provides methods to screen a compound for the ability to stimulate or inhibit interaction between the metalloprotease protein and a target molecule that normally interacts with the metalloprotease protein. The assay includes the steps of combining the metalloprotease protein with a candidate compound under conditions that allow the metalloprotease protein or fragment to interact with the target molecule, and to detect the formation of a complex between the metalloprotease protein and the target, or to detect the biochemical consequence of the interaction with the metalloprotease and the target.

In further related embodiments, the invention provides drug screening assays, in cell-based or cell-free systems. Cell-based systems can be native, i.e., cells that normally express the ADAMTS-12, as a biopsy, or expanded in cell culture. In one embodiment, cell-based assays involve recombinant host cells expressing ADAMTS-12. Accordingly, cells that are useful in this regard include, but are not limited to, cells differentially expressing ADAMTS-12 (e.g., metastasic cells). These include, but are not limited to, cells or tissues derived from an individual having an ADAMTS-12 disorder (e.g., cancerous tissue or tumors, synovial fluid). Such cells can naturally express the gene or can be recombinant. Recombinant cells include cells containing one or more copies of exogenously-introduced ADAMTS-12 sequences, or cells that have been genetically modified to modulate expression of the endogenous ADAMTS-12 sequence.

In these embodiments, the invention particularly relates to cells derived from subjects with disorders involving the tissues in which ADAMTS-12 is expressed or derived from tissues subject to disorders including, but not limited to, those disclosed herein. These disorders may naturally occur, as in populations of human subjects, or may occur in model systems such as in vitro systems or in vivo, such as in non-human transgenic organisms, particularly in non-human transgenic animals.

In yet another aspect of the invention, the invention provides methods to identify proteins that interact with the metalloprotease in the tissues and disorders disclosed. For example, the proteins of the invention can be used as “bait proteins” in a two-hybrid assay or three-hybrid assay (see, e.g., U.S. Pat. No. 5,283,317; Zervos et al. (1993) Cell 72:223-232; Madura et al. (1993) J. Blod. Chem. 268:12046-12054; Bartel et al. Biotechniques 14:920-924; Iwabuchi et al. (I 993) Oncogene 8:1693-1696; and Brent, WO 94/10300), to identify other proteins (captured proteins) which bind to or interact with the proteins of the invention and modulate their activity.

I. ADAMTS-12 Reagents

A. ADAM-TS Polypeptides

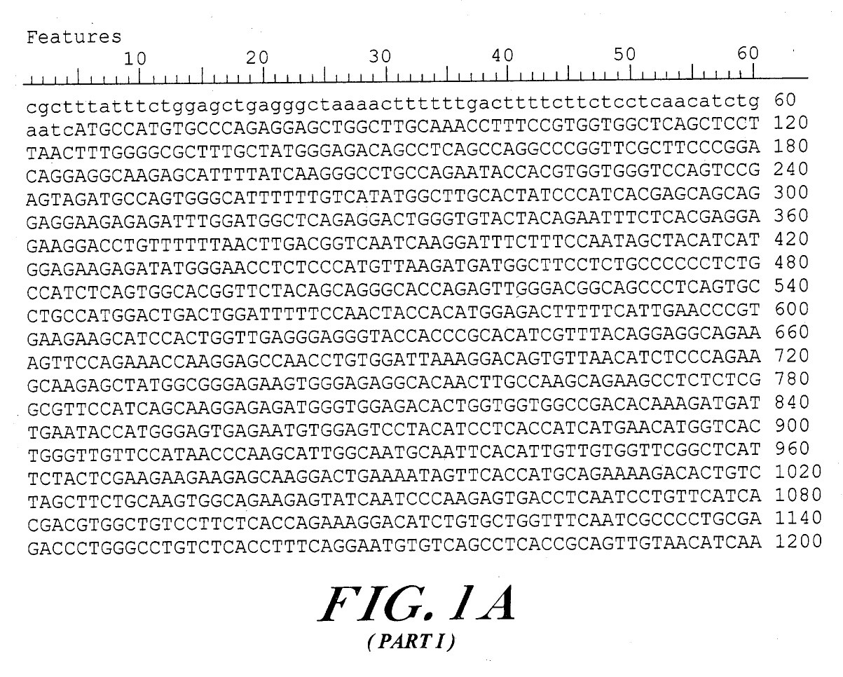

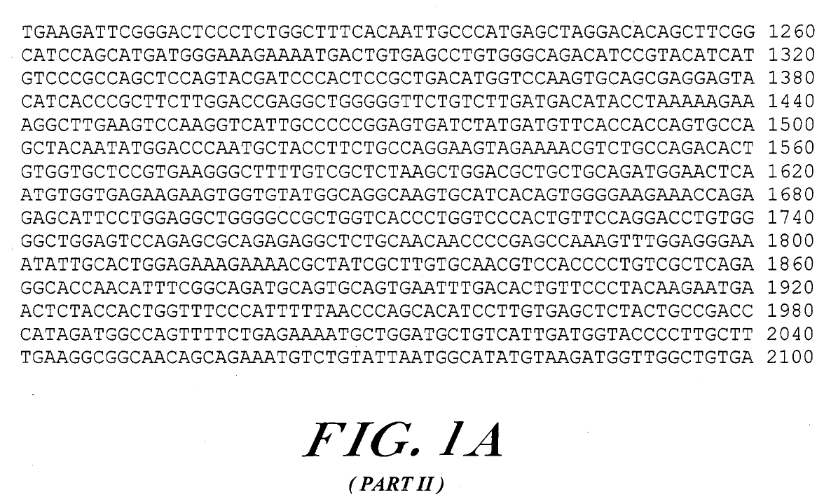

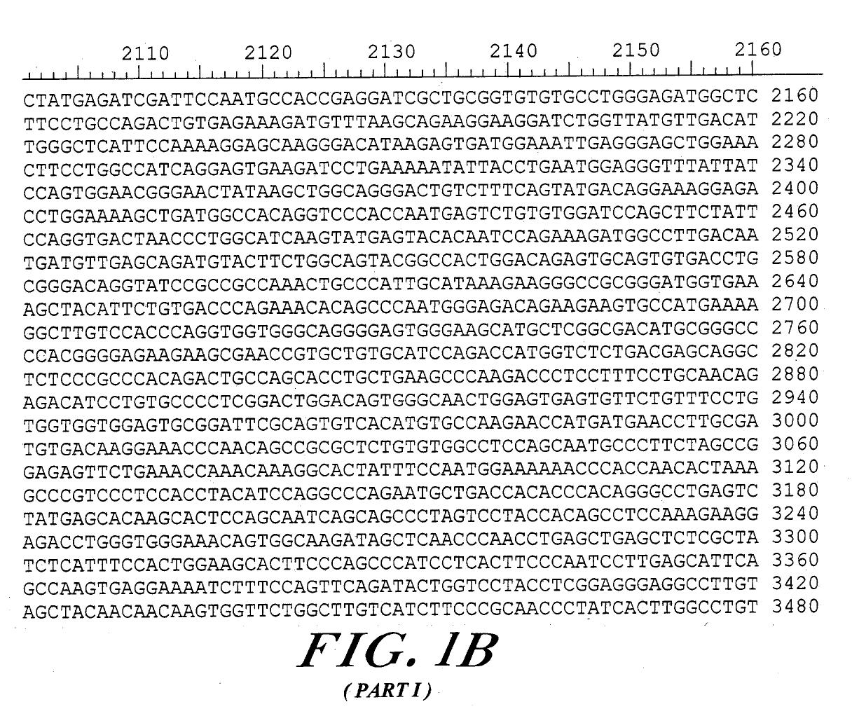

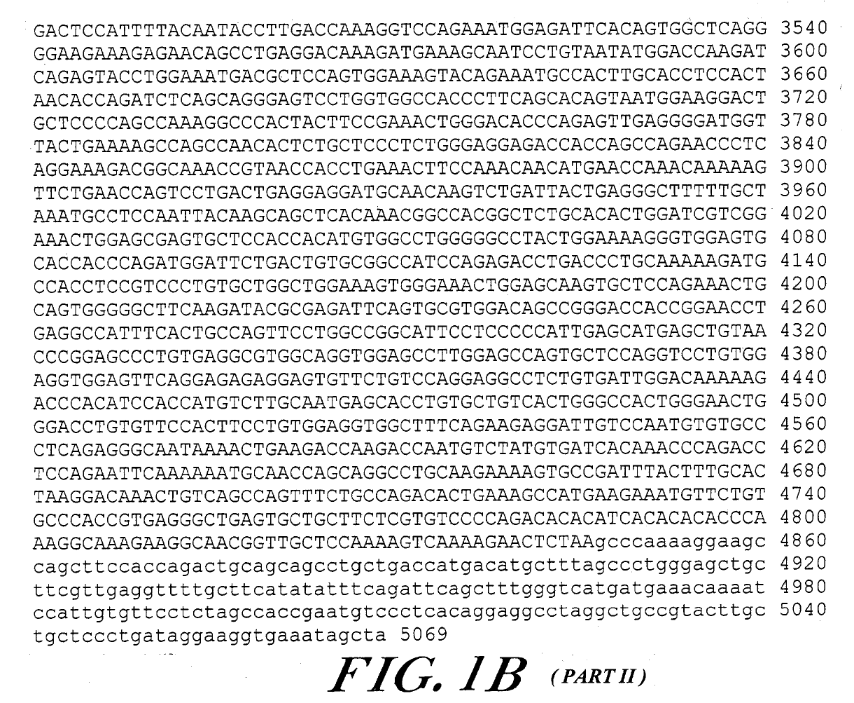

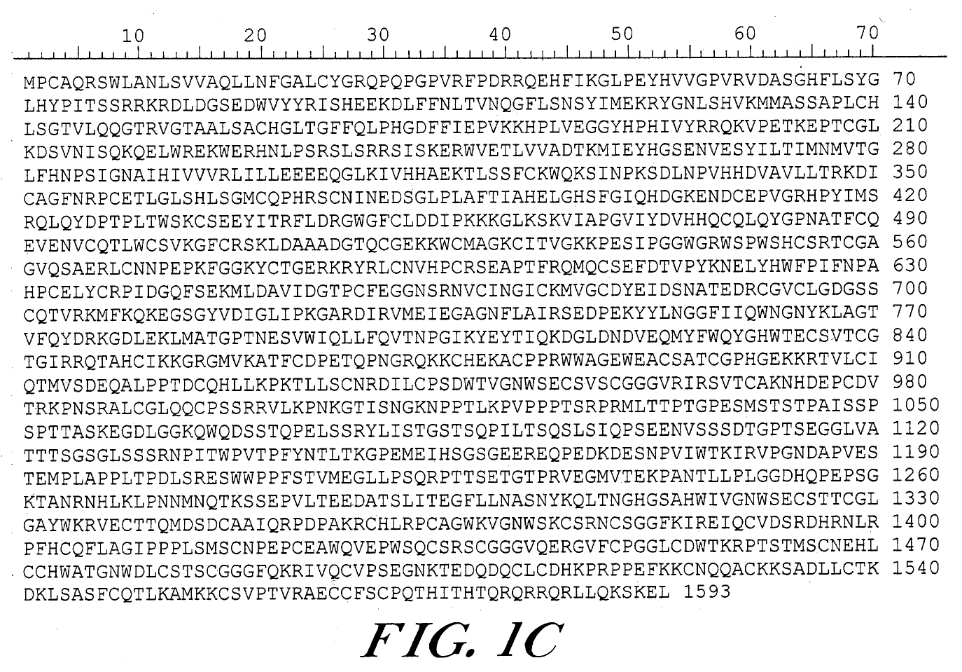

“ADAMTS-12 polypeptide” or “ADAMTS-12 protein” refers to the polypeptide in SEQ ID NO 1 (FIG. 1). This protein contains 1,543 amino acids with a predicted molecular mass of 177.5 kDa. Alignment of this sequence with sequences of the other members of the ADAM-TS family (FIG. 2) indicates that ADAMTS-12 is most similar to ADAM-TS7 (57% identity). ADAMTS-12 possesses all of the characteristic domains of the other ADAM-TS proteins including a signal sequence, propeptide, catalytic domain, disintegrin-like and cystein rich domains, as well as thrombospondin repeats (FIG. 3). However, while the first TS repeat is 52 amino acids, as in other ADAM-TS proteins, the organization of the remaining TS repeats are unique to this protein. All previously described human ADAM-TSs contain a second C-terminal TS module that is separated from the first TS repeat by a cysteine-rich domain and a spacer sequence. ADAMTS-12 contains three TS repeats in this second TS module, but, in addition, contains an third C-terminal TS module containing four TS repeats separated from the second TS module by a second spacer sequence 319 amino acids long. This TS module is then followed by a cysteine rich C-terminal domain, similar to C-terminal region of other ADAM-TSs.

Accordingly, the term “ADAMTS-12 protein” or “ADAMTS-12 polypeptide”, further includes fragments derived from the full-length ADAMTS-12s including various domains, as well as the numerous variants described herein.

The present invention thus utilizes an isolated or purified ADAMTS-12 polypeptide and variants and fragments thereof. As used herein, a polypeptide is said to be “isolated” or “purified” when it is substantially free of cellular material, when it is isolated from recombinant and non-recombinant cells, or free of chemical precursors or other chemicals when it is chemically synthesized. A polypeptide, however, can be joined to another polypeptide with which it is not normally associated in a cell and still be considered “isolated” or “purified.”

The ADAMTS-12 polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

In one embodiment, the language “substantially free of cellular material” includes preparations of ADAMTS-12 having less than about 30% (by dry weight) other proteins (i.e., contaminating protein), less than about 20% other proteins, less than about 10% other proteins, or less than about 5% other proteins. When the polypeptide is recombinantly produced, it can also be substantially free of culture medium, i.e., culture medium represents less than about 20%, less than about 10%, or less than about 5% of the volume of the protein preparation.

An ADAMTS-12 polypeptide is also considered to be isolated when it is part of a membrane preparation or is purified and then reconstituted with membrane vesicles or liposomes.

The language “substantially free of chemical precursors or other chemicals” includes preparations of ADAMTS-12 polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, the ADAMTS-12 polypeptide comprises the amino acid sequence shown in SEQ ID NO 1. However, the invention also encompasses sequence variants. Variants include a substantially homologous protein encoded by the same genetic locus in an organism, i.e., an allelic variant.

Variants also encompass proteins derived from other genetic loci in an organism, but having substantial homology to the ADAMTS-12 of SEQ ID NO 1. Variants also include proteins substantially homologous to ADAMTS-12 but derived from another organism, i.e., an ortholog. Variants also include proteins that are substantially homologous to ADAMTS-12 that are produced by chemical synthesis. Variants also include proteins that are substantially homologous to ADAMTS-12 that are produced by recombinant methods. It is understood, however, that variants exclude any amino acid sequences disclosed prior to the invention.

As used herein, two proteins (or a region of the proteins) are substantially homologous when the amino acid sequences are at least about 70-75%, typically at least about 80-85%, and most typically at least about 90-95%, 97%, 98% or 99% or more homologous. A substantially homologous amino acid sequence, according to the present invention, will be encoded by a nucleic acid sequence hybridizing to the nucleic acid sequence, or portion thereof, of the sequence shown in SEQ ID NO 2 under stringent conditions as more fully described below.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% or more of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The invention also encompasses polypeptides having a lower degree of identity but having sufficient similarity so as to perform one or more of the same functions performed by ADAMTS-12. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gin, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine Glycine |

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, HG., eds., Humana Press, New Jersey, 1994; Sequence Analysis in Molecular Biology, van Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al. (1993) Proc. Natl. Acad Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

In a preferred embodiment, the percent identity between two amino acid sequences is determined using the Needleman et al. (1970) (.I Mol. Biol. 48:444-453) algorithm which has been incorporated into the GAP program in the GCG software package using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (Devereux et al. (1984) Nucleic Acids Res. 12(1):387), using a NWSgapdna.CMP matrix and a gap weight of 40, 50, 60, 70, or 80 and a length weight of 1, 2, 3, 4, 5, or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC [GCG?]sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al. (1994) Comput Appl. Biosci. 10:3-5; and FASTA described in Pearson et al. (1988) PNAS 85:2444-8.

A variant polypeptide can differ in amino acid sequence by one or more substitutions, deletions, insertions, inversions, fusions, and truncations or a combination of any of these. Variant polypeptides can be fully functional or can lack function in one or more activities. Thus, in the present case, variations can affect the function, for example, of one or more of the regions corresponding to the prodomain, the metalloprotease catalytic region, the disintegrin region, the cysteine rich domain, carboxyterminal regulatory regions, aminoterminal regulatory regions, aminoterminal targeting regions, regions involved in membrane association, as well as regions involved in enzyme activation.

Fully functional variants typically contain only conservative variation or variation in non-critical residues or in noncritical regions. Functional variants can also contain substitution of similar amino acids, which results in no change or an insignificant change in function. Alternatively, such substitutions may positively or negatively affect function to some degree. The activity of such functional variants can be determined using metalloprotease, extracellular matrix, and cell motility assays that are standard in the art, such as those described herein.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for ADAMTS-12 polypeptide. This includes preventing immunogenicity from pharmaceutical formulations by preventing protein aggregation.

Useful variations further include alteration of catalytic activity. For example, one embodiment involves a variation at the binding site that results in binding but not metalloprotease activity. A further useful variation at the same site can result in altered affinity for substrate. Another useful variation provides a fusion protein in which one or more domains or subregions are operationally fused to one or more domains or subregions from another ADAM-TS isoform or family.

Substantial homology can be to the entire nucleic acid or amino acid sequence or to fragments of these sequences. The invention thus also includes polypeptide fragments of ADAMTS-12. Fragments can be derived from the amino acid sequence shown in SEQ ID NO 1. However, the invention also encompasses fragments of the variants of ADAMTS-12 as described herein.

Accordingly, a fragment can comprise at least about 10, 15, 20, 25, 30, 35, 40, 45, 50 or more contiguous amino acids. Fragments can retain one or more of the biological activities of the protein, for example the ability to bind to or hydrolyze peptide bonds, as well as fragments that can be used as an immunogen to generate ADAMTS-12 antibodies. Preferred antigenic regions of ADAMTS-12 protein are set fort, for example, in FIG. 4.

Biologically active fragments (peptides which are, for example, 5, 7, 10, 12, 15, 20, 30, 35, 36, 37, 38, 39, 40, 50, 100 or more amino acids in length) can comprise a domain or motif, e.g., a catalytic site, disintegrin domain, prodomain, etc. For example, the various domains of the ADAMTS-12 protein are set forth in FIGS. 2 and 4. As indicated, ADAMTS-12 contains a pre-pro region containing a signal sequence followed by a putative pro region containing three conserved cysteine residues. The C-terminal part of this region contains a consensus cleavage signal for furin that is cleaved to generate the mature protease.

The catalytic domain contains eight cysteine residues and a typical reprolysin-type zinc binding signature HEX1X2HX3X1GX1XHD (X is typically: 1, a hydrophobic residue, 2, glycine or a hydrophobic residue, 3, asparagine) which is involved in the coordination of the catalytic zinc atom at the active site. Five cysteine residues are upstream of the zinc binding sequence, while three residues are downstream. Like all metalloproteases and reprolysins, the zinc-binding signature is followed by a methionine residue located 16 amino acids C-terminal to the zinc-binding site that contributes to form the Met turn structure present in both reprolysins and matrix metalloproteases.

The catalytic domain is followed by a disintegrin-like domain of 79 residues with 30-45% similarity to snake venom disintegrins, but without the canonical cysteine arrangement seen in the latter. The disintegrin-like domain contains eight cysteine residues.

The first thrombospondin (TS) repeat is very similar in all of the ADAM-TSs and is 52 amino acids in length. This TS domain is followed by a conserved cysteine-rich sequence termed the cysteine-rich domain (CRD). It contains 10 conserved cysteines, and demonstrates high sequence homology with the CRD of other ADAMTS proteins.

As described above, unlike other ADAMTS proteins, ADAMTS-12 also contains a two spacer domains. The second spacer domain is 319 amino acid residues in length and lacks the sequence landmarks characteristic of the other domains. It also shows the least homology of all of the domains within the ADAMTS subfamily.

Accordingly useful figments of ADAMTS-12, for example, can extend in one or both directions from the functional sites or regions of the protein described herein to encompass 5, 10, 15, 20, 30, 40, 50, or up to 100 amino acids. Further, fragments can include sub-fragments of the specific domains mentioned above, which sub-fragments retain the function of the domain from which they are derived.

The invention also provides fragments with immunogenic properties. These contain an epitope-bearing portion of ADAMTS-12 and variants. These epitope-bearing peptides are useful to raise antibodies that bind specifically to a ADAMTS-12 polypeptide or region or fragment. These peptides can contain at least 10, 12, at least 14, or between at least about 15 to about 30 amino acids, and can be generated, for example, using the antigenic index profile of ADAMTS-12 (FIG. 4).

Non-limiting examples of antigenic polypeptides that can be used to generate antibodies include but are not limited to peptides derived from an extracellular site. However, intracellularly-made antibodies (“intrabodies”) are also encompassed, which would recognize intracellular peptide regions.

The epitope-bearing ADAMTS-12 polypeptides can be produced by any conventional means (Houghten, R. A. (1985) Proc. Natl. Acad. Sci. USA 82:5131-5135). Simultaneous multiple peptide synthesis is described in U.S. Pat. No. 4,631,211.

Fragments can be discrete (not fused to other amino acids or polypeptides) or can be within a larger polypeptide. Further, several fragments can be comprised within a single larger polypeptide. In one embodiment a fragment designed for expression in a host can have helerologous pre- and pro-polypeptide regions fused to the amino terminus of the ADAMTS-12 fragment and an additional region fused to the carboxyl terminus of the fragment.

The invention thus provides chimeric or fusion proteins. These comprise a ADAMTS-12 peptide sequence operatively linked to a heterologous peptide having an amino acid sequence not substantially homologous to the ADAMTS-12. “Operatively linked” indicates that the ADAMTS-12 peptide and the heterologous peptide are fused in-frame. The heterologous peptide can be fused to the N-terminus or C-terminus of ADAMTS-12 or can be internally located.

In one embodiment the fusion protein does not affect ADAMTS-12 function per se. For example, the fusion protein can be a GST-fusion protein in which the ADAMTS-12 sequences are fused to the N- or C-terminus of the GST sequences. Other types of fusion proteins include, but are not limited to, enzymatic fusion proteins, for example beta-galactosidase fusions, yeast two-hybrid GAL-4 fusions, poly-His fusions and Ig fusions. Such fusion proteins, particularly poly-His fusions, can facilitate the purification of recombinant ADAMTS-12. In certain host cells (e.g., mammalian host cells), expression and/or secretion of a protein can be increased by using a heterologous signal sequence. Therefore, in another embodiment, the fusion protein contains a heterologous signal sequence at its N-terminus.

EP-A-0 464533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists (Bennett et al. (1995) J: Mol. Recog. 8:52-58 (1995) and Johanson et al. J: Biol. Chem. 270:9459-9471). Thus, this invention also utilizes soluble fusion proteins containing a ADAMTS-12 polypeptide and various portions of the constant regions of heavy or light chains of immunoglobulins of various subclass (IgG, IgM, IgA, IgB). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence, which is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al. (1992) Current Protocols in Molecular Biology). Moreover, many expression vectors are commercially available that already encode a fusion moiety (e.g., a GST protein). An ADAMTS-12-encoding nucleic acid can be cloned into such an expression vector such that the fusion moiety is linked in-frame to ADAMTS-12.

Another form of fusion protein is one that directly affects ADAMTS-12 functions. Accordingly, an ADAMTS-12 polypeptide is encompassed by the present invention in which one or more of the ADAMTS-12 domains (or parts thereof) has been replaced by homologous domains (or parts thereof) from another ADAM family member. Accordingly, various permutations are possible. For example, the aminoterminal domain, or subregion thereof, can be replaced with the domain or subregion from another isoform or ADAM family. As a further example, the catalytic domain or parts thereof, can be replaced; the carboxyterminal domain or subregion can be replaced. Thus, chimeric ADAMTS-12s can be formed in which one or more of the native domains or subregions has been replaced by another.

Additionally, chimeric ADAMTS-12 proteins can be produced in which one or more functional sites is derived from a different isoform, or from another ADAM family. It is understood, however, that sites could be derived from ADAM families that occur in the mammalian genome but which have not yet been discovered or characterized.

The isolated ADAMTS-12 can be purified from cells that naturally express it, such as those demonstrating breakdown of extracellular matrix, increased cell motility or metastatsis among others, purified from cells that naturally express it but have been modified to overproduce ADAMTS-12, e.g., purified from cells that have been altered to express it (recombinant), synthesized using known protein synthesis methods, or by modifying cells that naturally encode ADAMTS-12 to express it.

In one embodiment, the protein is produced by recombinant DNA techniques. For example, a nucleic acid molecule encoding the ADAMTS-12 polypeptide is cloned into an expression vector, the expression vector introduced into a host cell and the protein expressed in the host cell. The protein can then be isolated from the cells by an appropriate purification scheme using standard protein purification techniques.

In other embodiments, the recombinant cell has been manipulated to activate expression of the endogenous ADAMTS-12 gene. For example, WO 99/15650 and WO 00/49162 describe a method of expressing endogenous genes termed, random activation of gene expression (RAGE), that can be used to activate or increase expression of endogenous ADAMTS-12. The RAGE methodology involves non-homologous recombination of a regulatory sequence to activate expression of a downstream endogenous gene. Alternatively, WO 94/12650, WO 95/31560, WO 96/29411, U.S. Pat. No. 5,733,761 and U.S. Pat. No. 6,270,985 describe a method of increasing expression of an endogenous gene that involves homologous recombination of a DNA construct that includes a targeting sequence, a regualtory sequence, an exon, and a splice-donor site. Upon homologous recombination a downsteam endogenous gene is expressed. The methods of expressing endogenous genes described in the forgoing patents are hereby expressely incorporated by reference.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd ed., T. E. Creighton, W. H. Freeman and Company, New York (1993). Many detailed reviews are available on this subject, such as by Wold, F., Posttranslational Covalent Modification of Proteins, B. C. Johnson. Ed., Academic Press, New York 1-12(1983); Seifter et al. (1990) Meth. Enzymol. 182: 626-646) and Rattan et al., (1992) Ann. NY: Acad. Sci. 663:48-62).

As is also well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the aminoterminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells, and, for this reason, insect cell expression systems have been developed to efficiently express mammalian proteins having native patterns of glycosylation. Similar considerations apply to other modifications. The same type of modification may be present in the same or varying degree at several sites in a given polypeptide. Also, a given polypeptide may contain more than one type of modification.

B. ADAM-TS Antibodies

The methods for using antibodies described above are based on the generation of antibodies that specifically bind to ADAMTS-12 or its variants or fragments.

To generate antibodies, an isolated ADAMTS-12 polypeptide is used as an immunogen to generate antibodies using standard techniques for polyclonal and monoclonal antibody preparation. Either the full-length protein, or one or more antigenic peptide fragments can be used.

Antibodies are preferably prepared from various regions of ADAMTS-12 described herein, or from discrete fragments in these regions. However, antibodies can be prepared from any region of the peptide as described herein. A preferred fragment produces an antibody that diminishes or completely prevents substrate binding. Antibodies can be developed against the entire ADAMTS-12 or domains of ADAMTS-12 as described herein. Antibodies can also be developed against specific functional sites as disclosed herein.

The antigenic peptide can comprise a contiguous sequence of at least 8, 9, 10, 12, 14, 15, or 30 amino acid residues. In one embodiment, fragments correspond to regions that are located on the surface of the protein, e.g., hydrophilic regions (FIG. 4). These fragments are not to be construed, however, as encompassing any fragments, which may be disclosed prior to the invention.

“Antibody” includes immunoglobulin molecules and immunologically active determinants of immunoglobulin molecules, i.e., molecules that contain an antigen binding site which specifically binds (immunoreacts with) an antigen. Structurally, the simplest naturally occurring antibody (e.g., IgG) comprises four polypeptide chains, two copies of a heavy (H) chain and two of a light (L) chain, all covalently linked by disulfide bonds. Specificity of binding in the large and diverse set of antibodies is found in the variable (V) determinant of the H and L chains; regions of the molecules that are primarily structural are constant (C) in this set. Antibody includes polyclonal antibodies, monoclonal antibodies, whole immunoglobulins, and antigen binding fragments of the immunoglobulins.

The binding sites of the proteins that comprise an antibody, i.e., the antigen-binding functions of the antibody, are localized by analysis of fragments of a naturally-occurring antibody. Thus, antigen-binding fragments are also intended to be designated by the term “antibody.” Examples of binding fragments encompassed within the term antibody include: a Fab fragment consisting of the V_{L}, V_{H}, C_{L} and C_{H1} domains; an F_{d} fragment consisting of the V_{H} and C_{H1} domains; an Fᵥ fragment consisting of the V_{L} and V_{H} domains of a single arm of an antibody; a dAb fragment (Ward et al., 1989 Nature 341:544-546) consisting of a V_{H} domain; an isolated complementarity determining region (CDR); and an F(ab′)₂ fragment, a bivalent fragment comprising two Fab′ fragments linked by a disulfide bridge at the hinge region. These antibody fragments are obtained using conventional techniques well-known to those with skill in the art, and the fragments are screened for utility in the same manner as are intact antibodies. The term “antibody” is further intended to include bispecific and chimeric molecules having at least one antigen binding determinant derived from an antibody molecule.

In the diagnostic and prognostic assays of the invention, the antibody can be a polyclonal antibody or a monoclonal antibody and in a preferred embodiment is a labeled antibody.

Polyclonal antibodies are produced by immunizing animals, usually a mammal, by multiple subcutaneous or intraperitoneal injections of an immunogen (antigen) and an adjuvant as appropriate. As an illustrative embodiment, animals are typically immunized against a protein, peptide or derivative by combining about 1 μg to 1 mg of protein capable of eliciting an immune response, along with an enhancing carrier preparation, such as Freund's complete adjuvant, or an aggregating agent such as alum, and injecting the composition intradermally at multiple sites. Animals are later boosted with at least one subsequent administration of a lower amount, as ⅕ to 1/10 the original amount of immunogen in Freund's complete adjuvant (or other suitable adjuvant) by subcutaneous injection at multiple sites. Animals are subsequently bled, serum assayed to determine the specific antibody titer, and the animals are again boosted and assayed until the titer of antibody no longer increases (i.e., plateaus).

Such populations of antibody molecules are referred to as “polyclonal” because the population comprises a large set of antibodies each of which is specific for one of the many differing epitopes found in the immunogen, and each of which is characterized by a specific affinity for that epitope. An epitope is the smallest determinant of antigenicity, which for a protein, comprises a peptide of six to eight residues in length (Berzofsky, J. and 1. Berkower, (1993) in Paul, W., Ed., Fundamental Immunology, Raven Press, N.Y., p.2⁴6). Affinities range from low, e.g. 10⁻⁶ M, to high, e.g., 10⁻¹¹ M. The polyclonal antibody fraction collected from mammalian serum is isolated by well known techniques, e.g. by chromatography with an affinity matrix that selectively binds immunoglobulin molecules such as protein A, to obtain the IgG fraction. To enhance the purity and specificity of the antibody, the specific antibodies may be further purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. Bound antibodies are eluted from the solid phase by standard techniques, such as by use of buffers of decreasing pH or increasing ionic strength, the eluted fractions are assayed, and those containing the specific antibodies are combined.

“Monoclonal antibody” or “monoclonal antibody composition” as used herein refers to a preparation of antibody molecules of single molecular composition. A monoclonal antibody composition displays a single binding specificity and affinity for a particular epitope. Monoclonal antibodies can be prepared using a technique which provides for the production of antibody molecules by continuous growth of cells in culture. These include but are not limited to the hybridoma technique originally described by Kohler and Milstein (1975, Nature 256:495497; see also Brown et al. 1981 J. Immunol 127:53946; Brown et al., 1980, J Biol Chem 255:4980-83; Yeh et al., 1976, PNAS 76:2927-31; and Yeh et al., 1982, Int. J. Cancer 29:269-75) and the more recent human B cell hybridoma technique (Kozbor et al., 1983, Immunol Today 4:72), EBV-hybridoma technique (Cole et al., 1985, Monoclonal Antibodies and Cancer Therapy, Alan R. Liss, Inc., pp. 77-96), and trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

A monoclonal antibody can be produced by the following steps. In all procedures, an animal is immunized with an antigen such as a protein (or peptide thereof) as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. A suspension of antibody-producing cells is removed from each immunized mammal secreting the desired antibody. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line. Mouse lymphocytes give a high percentage of stable fusions with mouse homologous myelomas, however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner are used with to standard techniques, for example, the P3-NS1/1-Ag4-1, P3-x63-Ag8.653 or Sp2/0-Ag14 myeloma lines, available from the American Type Culture Collection (ATCC), Rockville, Md.

The fusion-product cells, which include the desired hybridomas, are cultured in selective medium such as HAT medium, designed to eliminate unfused parental myeloma or lymphocyte or spleen cells. Hybridoma cells are selected and are grown under limiting dilution conditions to obtain isolated clones. The supernatants of each clonal hybridoma is screened for production of antibody of desired specificity and affinity, e.g., by immunoassay techniques to determine the desired antigen such as that used for immunization. Monoclonal antibody is isolated from cultures of producing cells by conventional methods, such as ammonium sulfate precipitation, ion exchange chromatography, and affinity chromatography (Zola et al., Monoclonal Hybridoma Antibodies: Techniques And Applications, Hurell (ed.), pp. 51-52, CRC Press, 1982). Hybridomas produced according to these methods can be propagated in culture in vitro or in vivo (in ascites fluid) using techniques well known to those with skill in the art.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening an antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu eta. (1987) Proc. Natl. Acad. Sci. USA 84:3439-3443; Liu et a. (1987) J. Immunol. 139:3521-3526; Sun et al. (1987) Proc. Natl. Acad. Sci. USA 84:214-218; Nishimura et al. (1987) Cancer Res. 47:999-1005; Wood el a. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Fully human antibodies can also be generated using transgenic mice in which the endogenous immunoglobulin genes have been inactivated and replaced with genes encoding the human light and heavy chain immunoglobulins. Such mice, and methods for using these mice to generate human polyclonal and monoclonal antibodies to an antigen are described for example in U.S. Pat. Nos. 6,075,181, 6,091,001 and 6,300,129, and in Tomizuka et al. (2000) Proc. Natl. Acad. Sci. 97:722-727.

“Labeled antibody” as used herein includes antibodies that are labeled by a detectable means and includes enzymatically, radioactively, fluorescently, chemiluminescently, and/or bioluminescently labeled antibodies.

One of the ways in which an antibody can be detectably labeled is by linking the same to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label the ADAMTS-12 specific antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection may be accomplished using any of a variety of immunoassays. For example, by radioactively labeling an antibody, it is possible to detect the antibody through the use of radioimmune assays. A description of a radioimmune assay (RIA) may be found in Laboratory Techniques and Biochemistry in Molecular Biology, by Work, T. S., et al., North Holland Publishing Company, NY (1978), with particular reference to the chapter entitled “An Introduction to Radioimmune Assay and Related Techniques” by Chard, T.

The radioactive isotope can be detected by such means as the use of a gamma counter or a scintillation counter or by audioradiography. Isotopes which are particularly useful for the purpose of the present invention are: ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthamide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

Likewise, a bioluminescent compound may be used to label an antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In the diagnostic and prognostic assays of the invention, the amount of binding of the antibody to the biological sample can be determined by the intensity of the signal emitted by the labeled antibody and/or by the number cells in the biological sample bound to the labeled antibody.

Antibodies directed toward a protein of interest can also be connected to magnetic beads and used to enrich a cell population. Immunomagnetic selection has been used previously for this purpose and examples of this method can be found, for example, at U.S. Pat. No. 5,646,001; Ree et al. (2002) Int. J. Cancer 97:28-33; Molnar et al. (2001) Clin. Cancer Research 7:4080-4085; and Kasimir-Bauer et al. (2001) Breast Cancer Res. Treat. 69:123-32. An antibody, either polyclonal or monoclonal, that is specific for a cell surface protein on a cell of interest is attached to a magnetic substrate thereby allowing selection of only those cells that express the surface protein of interest. Once a population of cells is selected, the following assays, can be performed to test for the presence of ADAMTS-12.

C. ADAM-TS Nucleic Acids

The invention further provides methods and uses for the nucleotide sequence in SEQ ID NO 2. The specifically disclosed cDNA comprises the coding region and 5′ and 3′ untranslated sequences in SEQ ID NO 2.

The invention provides isolated polynucleotides encoding ADAMTS-12. The term “ADAMTS-12 polynucleotide” or “ADAMTS-12 nucleic acid” refers to the sequences shown in SEQ ID NO 2. The term “ADAMTS-12 polynucleotide” o “ADAMTS-12 nucleic acid” further includes variants and fragments of the ADAMTS-12 polynucleotides.

An “isolated” ADAMTS-12 nucleic acid is one that is separated from other nucleic acid present in the natural source of the ADAMTS-12 nucleic acid. Preferably, an “isolated” nucleic acid is free of sequences which naturally flank the ADAMTS-12 nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB. The important point is that the ADAMTS-12 nucleic acid is isolated from flanking sequences such that it can be subjected to the specific manipulations described herein, such as recombinant expression, preparation of probes and primers, and other uses specific to the ADAMTS-12 nucleic acid sequences.

Moreover, an “isolated” nucleic acid molecule, such as a cDNA or RNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

The ADAMTS-12 polynucleotides can encode the mature protein plus additional amino or carboxyterminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The ADAMTS-12 polynucleotides include, but are not limited to, the sequence encoding the mature polypeptide alone, the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

ADAMTS-12 polynucleotides can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

In one embodiment, the ADAMTS-12 nucleic acid comprises only the coding region.

The invention further provides variant ADAMTS-12 polynucleotides, and fragments thereof, that differ from the nucleotide sequence shown in SEQ ID NO 2 due to degeneracy of the genetic code and thus encode the same protein as that encoded by the nucleotide sequence shown in SEQ ID NO 2.

The invention also provides ADAMTS-12 nucleic acid molecules encoding the variant polypeptides described herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Typically, variants have a substantial identity with a nucleic acid molecule of SEQ ID NO 2, and the complements thereof. Variation can occur in either or both the coding and non-coding regions. The variations can produce both conservative and non-conservative amino acid substitutions.

Orthologs, homologs, and allelic variants can be identified using methods well known in the art. These variants comprise a nucleotide sequence encoding a ADAMTS-12 that is at least about 60-65%, 65-70%, typically at least about 70-75%, more typically at least about 80-85%, and most typically at least about 90-95%, 96%, 97%, 98%, 99% or more homologous to the nucleotide sequence shown in SEQ ID NO 2 or a fragment of this sequence. Such nucleic acid molecules can readily be identified as being able to hybridize, under stringent conditions, to the nucleotide sequence shown in SEQ ID NO 2 or a fragment of the sequence. It is understood that stringent hybridization does not indicate substantial homology where it is due to general homology, such as poly A sequences, or sequences common to all or most proteins, or all cyclic nucleotide ADAMTS-12.

As used herein, the term “hybridizes under stringent conditions” is intended to describe conditions for hybridization and washing under which nucleotide sequences encoding a polypeptide at least about 60-65% homologous to each other typically remain hybridized to each other. The conditions can be such that sequences at least about 65%, at least about 70%, at least about 75%, at least about 80%, at least about 90%, at least about 95%, 96%, 97%, 98%, 99% or more identical to each other remain hybridized to one another. Such stringent conditions are known to those skilled in the art and can be found in Current Protocols in Molecular Biology, John Wiley & Sons, N.Y. (1989), 6.3.1-6.3.6, incorporated by reference. One example of stringent hybridization conditions are hybridization in 6× sodium chloride/sodium citrate (SSC) at about 45° C., followed by one or more washes in 0.2×SSC, 0.1% SDS at 50°, 55°, 60°, 62° or 65° C. In another non-limiting example, nucleic acid molecules are allowed to hybridize in 6× sodium chloride/sodium citrate (SSC) at about 45° C., followed by one or more low stringency washes in 0.2×SSC/0.1% SDS at room temperature, or by one or more moderate stringency washes in 0.2×SSC/0.1% SDS at 42° C., or washed in 0.2×SSC/0.1% SDS at 65° C. for high stringency. In one embodiment, an isolated nucleic acid molecule that hybridizes under stringent conditions to the sequence of SEQ ID NO 2.

As used herein, a “naturally-occurring” nucleic acid molecule refers to an RNA or DNA molecule having a nucleotide sequence that occurs in nature (e.g., encodes a natural protein).

As understood by those of ordinary skill, the exact conditions can be determined empirically and depend on ionic strength, temperature and the concentration of destabilizing agents such as formamide or denaturing agents such as SDS. Other factors considered in determining the desired hybridization conditions include the length of the nucleic acid sequences, base composition, percent mismatch between the hybridizing sequences and the frequency of occurrence of subsets of the sequences within other non-identical sequences. Thus, equivalent conditions can be determined by varying one or more of these parameters while maintaining a similar degree of identity or similarity between the two nucleic acid molecules.

The present invention also provides isolated nucleic acids that contain a single or double stranded fragment or portion that hybridizes under stringent conditions to the nucleotide sequence of SEQ ID NO 2 or the complement of SEQ ID NO 2. In one embodiment, the nucleic acid consists of a portion of the nucleotide sequence of SEQ ID NO 2 and the complement of SEQ ID NO 2. The nucleic acid fragments of the invention are at least about 15, preferably at least about 18, 20, 23 or 25 nucleotides, and can be 30, 40, 50, 60, 70, 80, 100, 150, 200, 250, 300, 350, 400, 450, 500, 600, 700, 800, 900, 1000 or more nucleotides in length. Longer fragments, for example, 30 or more nucleotides in length, which encode antigenic proteins or polypeptides described herein are useful.

Furthermore, the invention provides polynucleotides that comprise a fragment of the full-length ADAMTS-12 polynucleotide. The fragment can be single or double-stranded and can comprise DNA or RNA. The fragment can be derived from either the coding or the non-coding sequence.

In another embodiment an isolated ADAMTS-12 nucleic acid encodes the entire coding region. In another embodiment the isolated ADAMTS-12 nucleic acid encodes a sequence corresponding to the mature protein that may be from about amino acid 6 to the last amino acid. Other fragments include nucleotide sequences encoding the amino acid fragments described herein.

Thus, ADAMTS-12 nucleic acid fragments further include sequences corresponding to the domains described herein, subregions also described, and specific functional sites. ADAMTS-12 nucleic acid fragments also include combinations of the domains, segments, and other functional sites described above. A person of ordinary skill in the art would be aware of the many permutations that are possible.

Where the location of the domains or sites have been predicted by computer analysis, one of ordinary skill would appreciate that the amino acid residues constituting these domains can vary depending on the criteria used to define the domains.

However, it is understood that a ADAMTS-12 fragment includes any nucleic acid sequence that does not include the entire gene.

The invention also provides ADAMTS-12 nucleic acid fragments that encode epitope bearing regions of the ADAMTS-12 proteins described herein.

The nucleic acid fragments useful to practice the invention provide probes or primers in assays, such as those described herein. “Probes” are oligonucleotides that hybridize in a base-specific manner to a complementary strand of nucleic acid. Such probes include polypeptide nucleic acids, as described in Nielsen et al. (1991) Science 254: 1497-1500. Typically, a probe comprises a region of nucleotide sequence that hybridizes under highly stringent conditions to at least about 15, typically about 20-25, and more typically about 40, 50, 75 or 100 or more consecutive nucleotides of the nucleic acid sequence shown in SEQ ID NO 2 and the complements thereof. More typically, the probe further comprises a label, e.g., radioisotope, fluorescent compound, enzyme, or enzyme co-factor.

As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides. The term “primer site” refers to the area of the target DNA to which a primer hybridizes. The term “primer pair” refers to a set of primers including a 5′ (upstream) primer that hybridizes with the 5′ end of the nucleic acid sequence to be amplified and a 3′ (downstream) primer that hybridizes with the complement of the sequence to be amplified.

Where the polynucleotides are used to assess ADAMTS-12 properties or functions, such as in the assays described herein, all or less than all of the entire cDNA can be useful. Assays specifically directed to ADAMTS-12 functions, such as assessing agonist or antagonist activity, encompass the use of known fragments. Further, diagnostic methods for assessing ADAMTS-12 function can also be practiced with any fragment, including those fragments that may have been known prior to the invention. Similarly, in methods involving treatment of ADAMTS-12 dysfunction, all fragments are encompassed including those, which may have been known in the art.

The invention utilizes the ADAMTS-12 polynucleotides as a hybridization probe for cDNA and genomic DNA to isolate a full-length cDNA and genomic clones encoding variant polypeptides and to isolate cDNA and genomic clones that correspond to variants producing the same polypeptides shown in SEQ ID NO 1 or the other variants described herein. This method is useful for isolating variant genes and cDNA that are expressed in the cells, tissues, and disorders disclosed herein.

The probe can correspond to any sequence along the entire length of the gene encoding ADAMTS-12. Accordingly, it could be derived from 5′ noncoding regions, the coding region, and 3′ noncoding regions.

The nucleic acid probe can be, for example, the full-length cDNA of SEQ ID NO 2, or a fragment thereof, such as an oligonucleotide of at least 12, 15, 30, 50, 100, 250 or 500 nucleotides in length and sufficient to specifically hybridize under stringent conditions to mRNA or DNA.

Fragments of the polynucleotides can also be used to synthesize larger fragments or full-length polynucleotides described herein. For example, a fragment can be hybridized to any portion of an mRNA and a larger or full-length cDNA can be produced.

Fragments can also be used to synthesize antisense molecules of desired length and sequence.

Antisense nucleic acids, useful in treatment and diagnosis, can be designed using the nucleotide sequences of SEQ ID NO 2, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethy laminomethyl-2-thiouridine, 5-carboxymethy laminomethyl uracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl aminomethyl uracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

Additionally, the nucleic acid molecules useful to practice the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al (1996), supra; Perry-O'Keefe et al. (1996) Proc. Natl. Acad Sci. USA 93: 14670. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5: 1119.

The nucleic acid molecules and fragments useful to practice the invention can also include other appended groups such as peptides (e.g., for targeting host cell ADAMTS-12 in vivo), or agents facilitating transport across the cell membrane (see, e.g., Letsinger et al. (1989) Proc. Natl. Acad Sci. USA 86:6553-6556; Lemaitre et al. (1987) Proc. Natl. Acad. Sci. USA 84:648-652; PCT Publication No. WO 88/0918) or the blood brain barrier (see, e.g., PCT Publication No. WO 89/10134). In addition, oligonucleotides can be modified with hybridization-triggered cleavage agents (see, e.g., Krol et al. (1988) Bio-Techniques 6:958-976) or intercalating agents (see, e.g., Zon (1988) Pharm Res. 5:539-549).

D. Vectors and Host Cells

The invention also provides methods using vectors containing the ADAMTS-12 polynucleotides. The term “vector” refers to a vehicle, preferably a nucleic acid molecule that can transport the ADAMTS-12 polynucleotides. When the vector is a nucleic acid molecule, the ADAMTS-12 polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the ADAMTS-12 polynucleotides. Alternatively, the vector may integrate into the host cell genome to produce additional copies of the ADAMTS-12 polynucleotides when the host cell replicates, or to increase or activate expression of the endogenous ADAMTS-12 coding sequences.

The invention provides vectors for the maintenance (cloning vectors) or vectors for expression (expression vectors) of the ADAMTS-12 polynucleotides. The vectors can function in procaryotic or eukaryotic cells or in both (shuttle vectors).

Expression vectors contain cis-acting regulatory regions that are operably linked in the vector to the ADAMTS-12 polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the ADAMTS-12 polynucleotides from the vector. Alternatively, a trans-acting factor may be supplied by the host cell. Finally, a trans-acting factor can be produced from the vector itself.

It is understood, however, that in some embodiments, transcription and/or translation of the ADAMTS-12 polynucleotides can occur in a cell-free system.

The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV 40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al. (1989) Molecular Cloning: A Laboratory Manual 2nd ed, Cold Spring-Harbor Laboratory Press, Cold Spring Harbor, N.Y.).

A variety of expression vectors can be used to express a ADAMTS-12 polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV 40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al. (1989) Molecular Cloning: A Laboratory Manual 2nd ed., Cold Spring Harbor Laboratory Press, Cold Spring Harbor, N.Y.

The regulatory sequence may provide constitutive expression in one or more host cells (i.e., tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The ADAMTS-12 polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the ADAMTS-12 polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al. (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al (1988) Gene 69:301-315) and pET11d (Studier et al. (1990) Gene Expression Technology: Methods in Enzymology 185:60-89).

Recombinant protein expression can be maximized in a host bacteria by providing a genetic background wherein the host cell has an impaired capacity to proteolytically cleave the recombinant protein. (Gottesman, S. (1990) Gene Expression Technology: Methods in Enzymology 185, Academic Press, San Diego, Calif. 119-128). Alternatively, the sequence of the polynucleotide of interest can be altered to provide preferential codon usage for a specific host cell, for example E. coli. (Wada et al (1992) Nucleic Acids Res. 20:2111-2118).

The ADAMTS-12 polynucleotides can also be expressed by expression vectors that are operative in yeast. Examples of vectors for expression in yeast e.g., S. cerevisiae include pYepSec1 (Baldari et al. (1987) EMBO J: 6:229-234), pMFa (Kurjan et a. (1982) Cell 30:933-943), pJRY88 (Schultzei al. (1987) Gene 54:113-123), and pYES2 (Invitrogen Corporation, San Diego, Calif.).

The ADAMTS-12 polynucleotides can also be expressed in insect cells using, for example, baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow et a. (1989) Virology 170:31-39).

In certain embodiments of the invention, the polynucleotides described herein are expressed in mammalian cells using mammalian expression vectors. Examples of mammalian expression vectors include pCDM8 (Seed, B. (1987) Nature 329:840) and pMT2PC (Kaufman et al. (1987) EMBO J: 6:187-195).

The expression vectors listed herein are provided by way of example only of the well-known vectors available to those of ordinary skill in the art that would be useful to express the ADAMTS-12 polynucleotides. The person of ordinary skill in the art would be aware of other vectors suitable for maintenance propagation or expression of the polynucleotides described herein. These are found for example in Sambrook et al. (1989) Molecular Cloning: A Laboratory Manual 2nd; ed, Cold Spring Harbor Laboratory, Cold Spring Harbor Laboratory Press, Cold Spring Harbor, N.Y.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as' those found in Sambrook et al. (Molecular Cloning: A Laboratory Manual, 2d ed., Cold Spring Harbor Laboratory, Cold Spring Harbor Laboratory Press, Cold Spring Harbor, N.Y.). Host cells can contain more than one vector. Thus, different nucleotide sequences can be introduced on different vectors of the same cell. Similarly, the ADAMTS-12 polynucleotides can be introduced either alone or with other polynucleotides that are not related to the ADAMTS-12 polynucleotides such as those providing trans-acting factors for expression vectors. When more than one vector is introduced into a cell, the vectors can be introduced independently, co-introduced or joined to the ADAMTS-12 polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the ADAMTS-12 polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

In one embodiment the host cells of the present invention are cells that naturally produce ADAMTS-12 and have been modified to over produce the ADAMTS-12 polypeptide. This can be done, for example, by the technology known as RAGE, described in WO 99/15650 and WO 00/49162. RAGE involves randomly incorporating a transcriptional activator in the genome by non-homologous recombination, leading to activation or increased expression of genes down stream of the activator. Unlike other cloning methods the artisan needs no knowledge about the gene sequences. Further, the gene is expressed in cells that normally produce it rather than a host cell, e.g., E. coli. Once a RAGE modified cell has been selected, e.g., by activity, or phenotype, that cell can be cultured and used as an expression vector for the ADAMTS-12 polypeptide. This can also be done by a technology that relies on homologous recombination to incorporate a transcriptional activator into the genome, as described in WO 94//12650, WO 95/31560, and WO 96/29411, U.S. Pat. No. 5,733,761 and U.S. Pat. No. 6,270,985.

In addition to the vectors and host cells described above, the invention is intended to include cells which ADAMTS-12 naturally expressed, e.g., a cancer cell, or cells involved in extracellular matrix breakdown. These natural cells can be used in assays to determine the effectiveness of potential ADAMTS-12 modulators with regard to the invasiveness of a cell by the methods described herein.

II. Diagnostic and Prognostic Assays of the Invention

The diagnostic and prognostic methods of the present invention can be used to identify various types of disorders involving extracellular matrix breakdown and/or increased cell motility mediated by ADAMTS-12 (e.g., neoplastic, inflammatory and cardiovascular disorders), and is especially useful for detecting or predicting the onset of metastatic cancer. For example, ADAMTS-12 has been identified in gastrointestinal, colorectal, renal, lung and pancreatic carcinomas, and in Burkitt's lymphoma (Cal et al. (2001) J. Biol. Chem. 276:17932-17939)

As used herein, the term “cancer” refers to disorders characterized by deregulated or uncontrolled cell growth, for example, carcinomas, sarcomas, lymphomas. The term “cancer” includes benign tumors, primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “carcinoma” includes malignancies of epithelial or endocrine tissues, including respiratory system, gastrointestinal system, genitourinary system, testicles, breast, metastatic, cervix, lung, head and neck, colon and ovary, endocrine system, melanomas, and choriocarcinoma. The term “carcinoma” also includes cacinosarcomas, which include malignant tumors composed of carcinomatous and sarcomatous tissues. An “adenocarcinoma” revers to a carcinoma derived from glandular tissue or a tumor in which the tumor cells from recognizable glandular structures.

The terms “lymphoma” and “leukemia” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

“Anaplasia” refers to the histological features of cancer. These features include derangement of the normal tissue architecture, the crowding of cells, lack of cellular orientation termed dyspolarity, cellular heterogeneity in size and shape termed “pleomorphism.” The cytologic features of anaplasia include an increased nuclear-cytoplasmic ratio (the nuclear-cytoplasmic ratio can be over 50% for maligant cells), nuclear pleomorphism, clumping of the nuclear chromatin along the nuclear membrane, increased staining of the nuclear chromatin, simplified endoplasmic reticulum, increased free ribosomes, pleomorphism of mitochondria, decrease in size and number of organelles, enlarged and increased numbers of nucleoli, and sometimes the presence of intermediate filaments.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli which evoked the change. Neoplasms may show a partial or complete lack of structural organization and functional coordination with the normal tissue, and usually form a distinct mass of tissue. Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated.

By assessing the histologic and other features of a neoplasm, it can be determined whether the neoplasm is benign or malignant. Invasion and metastasis (the spread of the neoplasm to distant sites), as well as the breakdown of extracellular matrix are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. By contrast, malignant tumors generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical remove of benign tumors. By contrast, malignant tumors are locally invasive and grow into the adjacent tissues usually giving rise to irregular margins that are not encapsulated making it necessary to remove a wide margin of normal tissue for the surgical removal of malignant tumors. Benign neoplasms tends to grow more slowly than malignant tumors. Benign neoplasms also tend to be less autonomous than malignant tumors. Benign neoplasms tend to closely histologically resemble the tissue from which they originated. More high differentiated cancers, cancers that resemble the tissue from which they originated, tend to have a better prognosis than poorly differentiated cancers. Malignant tumors are more likely than benign tumors to have an aberrant function (i.e. the secretion of abnormal or excessive quantities of hormones).

“Invasive” or “aggressive” as used herein with respect to cancer refers to the proclivity of a tumor for expanding beyond its boundaries into adjacent tissue, or to the characteristic of the tumor with respect to metastasis (Darnell, J. (1990), Molecular Cell Biology, Third Ed., W.H. Freeman, NY). Invasive cancer can be contrasted with organ-confined cancer. For example, a basal cell carcinoma of the skin is a non-invasive or minimally invasive tumor, confined to the site of the primary tumor and expanding in size, but not metastasizing. In contrast, the cancer melanoma is highly invasive of adjacent and distal tissues. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

“Organ-confined” as used herein with cancer refers to cancer that has not metastasized beyond the boundaries of the particular organ in which it arose, i.e., has not been found by techniques familiar to those skilled in the art to occur in any organs or tissues. It can not be ruled out, however, that some number of cells have metastasized, however they are not detected by ordinary techniques used by those with skill in the art.

The term “metastasis” as used herein refers to the condition of spread of cancer from the organ of origin to additional distal sites in the patient. The process of tumor metastasis is a multistage event involving local invasion and destruction of intercellular and extracellular matrix, intravasation into blood vessels, lymphatics or other channels of transport, survival in the circulation, extravasation out of the vessels in the secondary site and growth in the new location (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zetter, N. Eng. J. Med. 322, 605-612 (1990)). Increased malignant cell motility has been associated with enhanced metastatic potential in animal as well as human tumors (Hosaka, et al., Gann 69, 273-276 (1978) and Haemmerlin, et al., Int. J. Cancer 27, 603-610 (1981)).

As used herein, the term “subject” includes living organisms; e.g., prokaryotes and eukaryotes. Examples of subjects include mammals, e.g., humans, dogs, cows, horses, kangaroos, pigs, sheep, goats, cats, mice, rabbits, rats, and transgenic non-human animals. Most preferably the subject is a human.

“Biological samples” include solid and body fluid samples. The biological samples of the present invention may include cells, protein or membrane extracts of cells, blood or biological fluids such as ascites fluid or brain fluid (e.g., cerebrospinal fluid). Examples of solid biological samples include samples taken from feces, the rectum, central nervous system, bone, breast tissue, renal tissue, the uterine cervix, the endometrium, the head/neck, the gallbladder, parotid tissue, the metastatic, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of “body fluid samples” include samples taken from the blood, serum, semen, metastatic fluid, seminal fluid, urine, saliva, sputum, mucus, bone marrow, lymph, and tears. For amplifying ADAMTS-12 RNA, the preferred samples include peripheral venous blood samples and metastatic tissue samples. Samples for use in the assays of the invention can be obtained by standard methods including venous puncture and surgical biopsy. In one embodiment, the biological sample is a metastatic tissue sample obtained by needle biopsy.

“Pharmacogenomics”, as used herein, refers to the application of genomics technologies such as gene sequencing, statistical genetics, and gene expression analysis to drugs in clinical development and on the market. More specifically, the term refers the study of how a patient's genes determine a subject's response to a drug (e.g., a patient's “drug response phenotype”, or “drug response genotype.”

A. Antibody Based Immunoassays

Methods for using antibodies as disclosed herein are particularly applicable to the cells, tissues and disorders that differentially express ADAMTS-12, or that are involved in metastasis and as otherwise discussed herein.

The invention provides methods using antibodies that selectively bind to ADAMTS-12 and its variants and fragments. An antibody is considered to selectively bind, even if it also binds to other proteins that are not substantially homologous with ADAMTS-12. These other proteins share homology with a fragment or domain of the ADAMTS-12. This conservation in specific regions gives rise to antibodies that bind to both proteins by virtue of the homologous sequence. In this case, it would be understood that antibody binding to ADAMTS-12 is still selective.

Antibodies accordingly can be used diagnostically to monitor protein levels in tissue as part of a clinical testing procedure, for example, to determine the efficacy of a given treatment regimen.

Additionally, antibodies are useful in pharmacogenomic analysis. Thus, antibodies prepared against polymorphic ADAMTS-12 can be used to identify individuals that require modified treatment modalities.

Antibodies can also be used in diagnostic procedures as an immunological marker for aberrant ADAMTS-12 analyzed by electrophoretic mobility, isoelectric point, tryptic peptide digest, and other physical assays known to those in the art.

Antibody detection of circulating fragments of the full length ADAMTS-12 can be used to identify ADAMTS-12 turnover.

Further, the antibodies can be used to assess ADAMTS-12 expression in disease states such as in active stages of the disease or in an individual with a predisposition toward disease related to ADAMTS-12 function. When a disorder is caused by an inappropriate tissue distribution, developmental expression, or level of expression of ADAMTS-12 protein, the antibody can be prepared against the normal ADAMTS-12 protein. If a disorder is characterized by a specific mutation in ADAMTS-12, antibodies specific for this mutant protein can be used to assay for the presence of the specific mutant ADAMTS-12. However, intracellularly-made antibodies (“intrabodies”) are also encompassed, which would recognize intracellular ADAMTS-12 peptide regions.

The antibodies can also be used to assess normal and aberrant subcellular localization in cells in the various tissues in an organism. Antibodies can be developed against the whole ADAMTS-12 or portions of ADAMTS-12.

The amount of an antigen (i.e. ADAMTS-12) in a biological sample may be determined by a radioimmunoassay, an immunoradiometric assay, and/or an enzyme immunoassay.

“Radioimmunoassay” is a technique for detecting and measuring the concentration of an antigen using a labeled (i.e. radioactively labeled) form of the antigen. Examples of radioactive labels for antigens include ³H, ¹⁴C, and ¹²⁵I. The concentration of antigen (i.e. ADAMTS-12) in a sample (i.e. biological sample) is measured by having the antigen in the sample compete with a labeled (i.e. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” The “Enzyme-Linked Immunosorbent Assay (ELISA)” is a technique for detecting and measuring the concentration of an antigen using a labeled (i.e. enzyme linked) form of the antibody.

In a “sandwich ELISA”, an antibody (i.e. to ADAMTS-12) is linked to a solid phase (i.e. a microtiter plate) and exposed to a biological sample containing antigen (i.e. ADAMTS-12). The solid phase is then washed to remove unbound antigen. A labeled (i.e. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be assayed for.

In a “competitive ELISA”, antibody is incubated with a sample containing antigen (i.e. ADAMTS-12). The antigen-antibody mixture is then contacted with an antigen-coated solid phase (i.e. a microtiter plate). The more antigen present in the sample, the less free antibody that will be available to bind to the solid phase. A labeled (i.e. enzyme linked) secondary antibody is then added to the solid phase to determine the amount of primary antibody bound to the solid phase.

In a “immunohistochemistry assay” a section of tissue for is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or β-galactosidase), or chemical methods (e.g., DAB/Substrate chromagen).

B. ADAMTS-12 Nucleic Acid-Based Diagnostic and Prognostic Methods

Also encompassed by this invention is a method of diagnosing ADAMTS-12 related disorders in a subject, comprising: detecting a level of ADAMTS-12 nucleic acid in a biological sample; and comparing the level of ADAMTS-12 in the biological sample with a level of ADAMTS-12 in a control sample, wherein an elevation in the level of ADAMTS-12 in the biological sample compared to the control sample is indicative of ADAMTS-12 disorder.

In addition, this invention pertains to a method of diagnosing ADAMTS-12 disorder metastasis in a subject, comprising the steps of: detecting a level of ADAMTS-12 nucleic acid in a biological sample; and comparing the level of ADAMTS-12 in the biological sample with a level of ADAMTS-12 in a control sample, wherein an elevation in the level of ADAMTS-12 in the biological sample compared to the control sample is indicative of an ADAMTS-12 disorder In preferred embodiments the ADAMTS-12 disorder is cancer, arthritis (i.e., rheumatoid arthritis, osteoarthritis) or cardiovasuclar disease. In a particular embodiment the disorder is metastatic cancer.

In an embodiment of the above methods, the detecting a level of ADAMTS-12 nucleic acid in a biological sample includes amplifying ADAMTS-12 RNA. In another embodiment of the above methods, the detecting a level of ADAMTS-12 nucleic acid in a biological sample includes hybridizing the ADAMTS-12 RNA with a probe.

As an alternative to making determinations based on the absolute expression level of the ADAMTS-12 marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-metastatic cancer sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the biological sample (absolute level of expression) is then divided by the mean expression value obtained for that marker. This provides a relative expression level.

One preferred diagnostic method for the detection of mRNA levels involves contacting the isolated mRNA with a nucleic acid molecule (probe) that can hybridize to the mRNA encoded by the gene being detected. Probes based on the sequence of a nucleic acid molecule of the invention can be used to detect transcripts corresponding to ADAMTS-12. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Hybridization of an mRNA with the probe indicates that the marker in question is being expressed. In an embodiment, the probe includes a label group attached thereto, e.g., a radioisotope, a fluorescent compound, an enzyme, or an enzyme co-factor.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

“Amplifying” refers to template-dependent processes and vector-mediated propagation which result in an increase in the concentration of a specific nucleic acid molecule relative to its initial concentration, or in an increase in the concentration of a detectable signal. As used herein, the term template-dependent process is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, J. D. et al., In: Molecular Biology of the Gene, 4th Ed., W. A. Benjamin, Inc., Menlo Park, Calif. (1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by Cohen et al. (U.S. Pat. No. 4,237,224), Maniatis, T. et al., Molecular Cloning (A Laboratory Manual), Cold Spring Harbor Laboratory, 1982.

A number of template dependent processes are available to amplify the target sequences of interest present in a sample. One of the best known amplification methods is the polymerase chain reaction (PCR) which is described in detail in Mullis, et al., U.S. Pat. No. 4,683,195, Mullis, et al., U.S. Pat. No. 4,683,202, and Mullis, et al., U.S. Pat. No. 4,800,159, and in Innis et al., PCR Protocols, Academic Press, Inc., San Diego Calif., 1990. Briefly, in PCR, two primer sequences are prepared which are complementary to regions on opposite complementary strands of the target sequence. An excess of deoxynucleoside triphosphates are added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction products and the process is repeated. Preferably a reverse transcriptase PCR amplification procedure may be performed in order to quantify the amount of mRNA amplified. Polymerase chain reaction methodologies are well known in the art.

Another method for amplification is the ligase chain reaction (LCR), disclosed in European Patent No. 320,308B1. In LCR, two complementary probe pairs are prepared, and in the presence of the target sequence, each pair will bind to opposite complementary strands of the target such that they abut In the presence of a ligase, the two probe pairs will link to form a single unit. By temperature cycling, as in PCR, bound ligated units dissociate from the target and then serve as “target sequences” for ligation of excess probe pairs. Whiteley, et al., U.S. Pat. No. 4,883,750 describes an alternative method of amplification similar to LCR for binding probe pairs to a target sequence.

Qbeta Replicase, described in PCT Application No. PCT/US87/00880 may also be used as still another amplification method in the present invention. In this method, a replicative sequence of RNA which has a region complementary to that of a target is added to a sample in the presence of an RNA polymerase. The polymerase will copy the replicative sequence which can then be detected.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

ADAMTS-12 specific sequences can also be detected using a cyclic probe reaction (CPR). In CPR, a probe having a 3′ and 5′ sequences of non-metastatic specific DNA and middle sequence of metastatic specific RNA is hybridized to DNA which is present in a sample. Upon hybridization, the reaction is treated with RNaseH, and the products of the probe identified as distinctive products generating a signal which are released after digestion. The original template is annealed to another cycling probe and the reaction is repeated. Thus, CPR involves amplifying a signal generated by hybridization of a probe to a metastatic cancer specific expressed nucleic acid.

Still other amplification methods described in GB Application No. 2 202 328, and in PCT Application No. PCT/US89/01025 may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh D., et al., Proc. Natl. Acad. Sci. (U.S.A.) 1989, 86:1173, Gingeras T. R., et al., PCT Application WO 88/1D315), including nucleic acid sequence based amplification (NASBA) and 3SR. In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has metastatic specific sequences. Following polymerization, DNA/RNA hybrids are digested with RNase H while double stranded DNA molecules are heat denatured again. In either case the single stranded DNA is made fully double stranded by addition of second metastatic specific primer, followed by polymerization. The double stranded DNA molecules are then multiply transcribed by a polymerase such as T7 or SP6. In an isothermal cyclic reaction, the RNAs are reverse transcribed into double stranded DNA, and transcribed once against with a polymerase such as T7 or SP6. The resulting products, whether truncated or complete, indicate metastatic cancer specific sequences.

Davey, C., et al., European Patent No. 329,822B1 disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H(RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller, H. I., et al., PCT Application WO 89/06700 discloses a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “race” disclosed by Frohman, M. A., In: PCR Protocols: A Guide to Methods and Applications 1990, Academic Press, New York) and “one-sided PCR” (Ohara, O., et al., Proc. Natl. Acad. Sci. (U.S.A.) 1989, 86:5673-5677).

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi, et al. (1989) Bio Technology 6:11.97) or any other nucleic acid amplification method, followed by the detection of the amplified molecules using techniques well-known to those of skill in the art. These detection schemes are especially useful for the detection of nucleic acid molecules if such molecules are present in very low numbers.

Methods based on ligation of two (or more) oligonucleotides in the presence of nucleic acid having the sequence of the resulting “di-oligonucleotide”, thereby amplifying the di-oligonucleotide (Wu, D. Y. et al., Genomics 1989, 4:560), may also be used in the amplification step of the present invention.

Following amplification, the presence or absence of the amplification product may be detected. The amplified product may be sequenced by any method known in the art, including and not limited to the Maxam and Gilbert method. The sequenced amplified product is then compared to a sequence known to be in a metastatic cancer specific sequence. Alternatively, the nucleic acids may be fragmented into varying sizes of discrete fragments. For example, DNA fragments may be separated according to molecular weight by methods such as and not limited to electrophoresis through an agarose gel matrix. The gels are then analyzed by Southern hybridization. Briefly, DNA in the gel is transferred to a hybridization substrate or matrix such as and not limited to a nitrocellulose sheet and a nylon membrane. A labeled probe is applied to the matrix under selected hybridization conditions so as to hybridize with complementary DNA localized on the matrix. The probe may be of a length capable of forming a stable duplex. The probe may have a size range of about 200 to about 10,000 nucleotides in length, preferably about 200 nucleotides in length. Various labels for visualization or detection are known to those of skill in the art, such as and not limited to fluorescent staining, ethidium bromide staining for example, avidin/biotin, radioactive labeling such as ³²P labeling, and the like. Preferably, the product, such as the PCR product, may be run on an agarose gel and visualized using a stain such as ethidium bromide. The matrix may then be analyzed by autoradiography to locate particular fragments which hybridize to the probe.

The ADAMTS-12 polynucleotides are also useful for monitoring the effectiveness of modulating compounds on the expression or activity of the ADAMTS-12 gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

Monitoring can be, for example, as follows: (i) obtaining a pre-administration sample from a subject prior to administration of the agent; (ii) detecting the level of expression of a specified mRNA or genomic DNA of the invention in the pre-administration sample; (iii) obtaining one or more post-administration samples from the subject; (iv) detecting the level of expression or activity of the mRNA or genomic DNA in the post-administration samples; (v) comparing the level of expression or activity of the mRNA or genomic DNA in the pre-administration sample with the mRNA or genomic DNA in the post-administration sample or samples; and (vi) increasing or decreasing the administration of the agent to the subject accordingly.

The ADAMTS-12 polynucleotides can be used in diagnostic assays for qualitative changes in ADAMTS-12 nucleic acid, and particularly in qualitative changes that lead to pathology. The polynucleotides can be used to detect mutations in ADAMTS-12 genes and gene expression products such as mRNA. The polynucleotides can be used as hybridization probes to detect naturally-occurring genetic mutations in the ADAMTS-12 gene and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of the ADAMTS-12 gene associated with a dysfunction provides a diagnostic tool for an active disease or susceptibility to disease when the disease results from overexpression, underexpression, or altered expression of ADAMTS-12.

Mutations in the ADAMTS-12 gene can be detected at the nucleic acid level by a variety of techniques. Genomic DNA can be analyzed directly or can be amplified by using PCR prior to analysis. RNA or cDNA can be used in the same way.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241: 1077-1080; and Nakazawa et al. (1994) PNAS 91:360-364), the latter of which can be particularly useful for detecting point mutations in the gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

Alternatively, mutations in a ADAMTS-12 gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site.

Perfectly matched sequences can be distinguished from mismatched sequences by nuclease cleavage digestion assays or by differences in melting temperature.

Sequence changes at specific locations can also be assessed by nuclease protection assays such as RNase and SI protection or the chemical cleavage method.

Furthermore, sequence differences between a mutant ADAMTS-12 gene and a wild-type gene can be determined by direct DNA sequencing. A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromalogr. 36:127-162; and Griffin et al. (1993) Appl. Biochem. Biotechnol. 38:147-159).

Other methods for detecting mutations in the gene include methods in which protection from cleavage agents is used to detect mismatched bases in RNA/RNA or RNA/DNA duplexes (Myers et al. (1985) Science 230: 1242); Cotton et al. (1988) PNAS 85:4397; Saleeba et al. (1992) Meth. Enzymol. 217:286-295), electrophoretic mobility of mutant and wild type nucleic acid is compared (Orita et al (1989) PNAS 86:2766; Cotton et al. (1993) Mutat. Res. 285:125-144; and Hayashi et al. (1992) Genet. Anal. Tech. Appl. 9:73-79), and movement of mutant or wild-type fragments in polyacrylamide gels containing a gradient of denaturant is assayed using denaturing gradient gel electrophoresis (Myers et al. (1985) Nature 313:495). The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7: 5). Examples of other techniques for detecting point mutations include, selective oligonucleotide hybridization, selective amplification, and selective primer extension.

In other embodiments, genetic mutations can be identified by hybridizing a sample and control nucleic acids, e.g., DNA or RNA, to high density arrays containing hundreds or thousands of oligonucleotide probes (Cronin et al (1996) Human Mutation 7:244-255; Kozal et al. (1996) Nature Medicine 2:753-759). For example, genetic mutations can be identified in two dimensional arrays containing light-generated DNA probes as described in Cronin et al supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

The ADAMTS-12 polynucleotides can also be used for testing an individual for a genotype that while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the ADAMTS-12 gene that results in altered affinity for substrate could result in an excessive or decreased drug effect with standard concentrations substrate. Accordingly, the ADAMTS-12 polynucleotides described herein can be used to assess the mutation content of the gene in an individual in order to select an appropriate compound or dosage regimen for treatment.

Thus polynucleotides displaying genetic variations that affect treatment provide a diagnostic target that can be used to tailor treatment in an individual. Accordingly, the production of recombinant cells or animals containing these polymorphisms allow effective clinical design of treatment compounds and dosage regimens.

The methods can involve obtaining a control biological sample from a control subject, contacting the control sample with a compound or agent capable of detecting mRNA, or genomic DNA, such that the presence of mRNA or genomic DNA is detected in the biological sample, and comparing the presence of mRNA or genomic DNA in the control sample with the presence of mRNA or genomic DNA in the test sample.

III. Methods for Identifying ADAMTS-12 Modulators

Determining the ability of the metalloprotease to bind to a target molecule can also be accomplished using a technology such as real-time Bimolecular Interaction Analysis (BIA). Sjolander et al. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the one-bead one-compound library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckerman et al. (1994) J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13: 412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. U.S.A. 97:6378-6382); Felici (1991) J. Mol. Biol. 222:301-310); (Ladner supra).

Candidate compounds include, for example, 1) peptides such as soluble peptides, including Ig-tailed fusion peptides and members of random peptide libraries (see, e.g., Lam et al. (19911) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble full-length metalloprotease or fragment that competes for substrate. Other candidate compounds include mutant metalloprotease or approaching fragments containing mutations that affect metalloprotease function and thus compete for substrate. Accordingly, a fragment that competes for substrate, for example with a higher affinity, or a fragment that binds substrate but does not hydrolyze it, is encompassed by the invention.

Protein inhibitors of the present invention can be selected using the RNA-protein fusion method that was developed by Szostak, J. W., et al. This method relies on a covalent fusion between an mRNA and a protein or peptide that it encodes through a puromycin at the 3′ end of the RNA molecule. Fusion of the polypeptide to the RNA that encodes it allows for the skilled artisan to isolate a protein of interest while also isolating the nucleic acid that encodes the protein. The technology is described in Roberts, R. W. and Szostak, J. W. (1997) Prot. Natl. Acad. Sci. USA 11:12297-302 and Liu, R. et al. (2000) Methods Enzymol. 318:268-93, and in U.S. Pat. Nos. 6,207,446, 6,214,553, 6,261,804, 6,258,558, and 6,281,344.

The invention provides other end points to identify compounds that modulate (stimulate or inhibit) ADAMTS-12 activity. The assays typically involve an assays that indicate ADAMTS-12 activity. Thus, the expression of genes that are up- or down-regulated in response to the metalloprotease dependent signal cascade can be assayed. In one embodiment, the regulatory region of such genes can be operably linked to a marker that is easily detectable, such as luciferase. Alternatively, cleavage of the metalloprotease target could also be measured.

Any of the biological or biochemical functions mediated by the metalloprotease can be used as an endpoint assay. These include all of the biochemical or biochemical/biological events described herein, in the references cited herein, incorporated by reference for these endpoint assay targets, and other functions known to those of ordinary skill in the art.

Assays for metalloproteases are common in the field and have been previously described. For example, a stromelysin assay can be used to assay a metalloprotease in the presence of inhibitors (Doughty, et al. (1993) Agents Actions 39:Cl 51-3), or the α₂-macroglobulin proteinase trapping assay (Kuno, et al. (1999) J. Biol. Chem. 274:18821-6; Loechel, F et al. (1999) J. Biol. Chem. 274:13427-33; Cal, S. et al. (2001) J. Biol. Chem. 276:1793240) can be used to measure the protease activity of ADAM polypeptides.

The invention provides competition binding assays designed to discover compounds that interact with the metalloprotease. Thus, a compound is exposed to a metalloprotease polypeptide under conditions that allow the compound to bind or to otherwise interact with the polypeptide. Soluble ADAMTS-12 polypeptide is also added to the mixture. If the test compound interacts with the soluble ADAMTS-12 polypeptide, it decreases the amount of complex formed or activity from the ADAMTS-12 target. This type of assay is particularly useful in cases in which compounds are sought that interact with specific regions of the metalloprotease. Thus, the soluble polypeptide that competes with the target metalloprotease region is designed to contain peptide sequences corresponding to the region of interest.

Another type of competition-binding assay can be used to discover compounds that interact with specific functional sites. Accordingly, compounds can be discovered that directly interact with ADAMTS-12 and compete with substrate. Such assays can involve any other component that interacts with ADAMTS-12.

To perform cell-free drug screening assays, it is desirable to immobilize either the ADAMTS-12, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/ADAMTS-12 fusion proteins can be absorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes is dissociated. Alternatively, the complexes can be dissociated from the matrix, separated by SDS-PAGE, and the level of ADAMTS-12-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a ADAMTS-12-binding target component, and a candidate compound are incubated in the ADAMTS-12-presenting wells and the amount of complex trapped in the well can be quantitated. Methods for detecting such complexes, in addition to those described above for the GST-immobilized complexes, include immunodetection of complexes using antibodies reactive with the ADAMTS-12 target molecule, or which are reactive with ADAMTS-12 and compete with the target molecule; as well as enzyme-linked assays which rely on detecting an enzymatic activity associated with the target molecule.

Nucleic acid expression assays are also useful for drug screening to identify compounds that modulate ADAMTS-12 nucleic acid expression (e.g., antisense, polypeptides, peptidomimetics, small molecules or other drugs). A cell is contacted with a candidate compound and the expression of mRNA determined. The level of expression of the mRNA in the presence of the candidate compound is compared to the level of expression of the mRNA in the absence of the candidate compound. The candidate compound can then be identified as a modulator of nucleic acid expression based on this comparison and be used, for example to treat a disorder characterized by aberrant nucleic acid expression. The modulator can bind to the nucleic acid or indirectly modulate expression, such as by interacting with other cellular components that affect nucleic acid expression.

Modulatory methods can be performed in vitro (e.g., by culturing the cell with the agent) or, alternatively, in vivo (e.g., by administering the gene to a subject) in patients or in transgenic animals.

The invention thus provides a method for identifying a compound that can be used to treat a disorder associated with expression of the ADAMTS-12 gene. The method typically includes assaying the ability of the compound to modulate the expression of the ADAMTS-12 nucleic acid and thus identifying a compound that can be used to treat a disorder characterized by excessive or deficient ADAMTS-12 nucleic acid expression.

The assays can be performed in cell-based and cell-free systems, such as systems using the tissues described herein, in which the gene is expressed or in model systems for the disorders to which the invention pertains. Cell-based assays include cells naturally expressing the ADAMTS-12 nucleic acid or recombinant cells genetically engineered to express specific nucleic acid sequences.

Alternatively, candidate compounds can be assayed in vivo in patients or in transgenic animals. The assay for ADAMTS-12 nucleic acid expression can involve direct assay of nucleic acid levels, such as mRNA levels.

Thus, modulators of ADAMTS-12 gene expression can be identified in a method wherein a cell is contacted with a candidate compound and the expression of mRNA determined. The level of expression of ADAMTS-12 mRNA in the presence of the candidate compound is compared to the level of expression of ADAMTS-12 mRNA in the absence of the candidate compound. The candidate compound can then be identified as a modulator of nucleic acid expression based on this comparison and be used, for example, to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

IV. ADAMTS-12 Cell Assays and Trangenic Animal Models

The methods using vectors and host cells described herein are useful where the host cells are those that naturally express the gene and which may be the native or a recombinant cell expressing the gene. The host cells of the present invention are useful for identifying compounds that modulate ADAMTS-12 activity, as well as for testing the toxcity of compounds identified to modulate ADAMTS-12.

It is understood that “host cells” and “recombinant host cells” refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

The host cells expressing the polypeptides described herein, and particularly recombinant host cells, have a variety of uses. First, the cells are useful for producing ADAMTS-12 proteins or polypeptides that can be further purified to produce desired amounts of ADAMTS-12 protein or fragments. Thus, host cells containing expression vectors are useful for polypeptide production, as well as cells producing significant amounts of the polypeptide.

In one embodiment, host cells of the invention include cells that naturally produce ADAMTS-12, e.g., a tumor cell. ADAMTS-12 has been shown to be produced in kidney, skeletal muscle, spleen, fetal liver, heart, stomach, uterus, salivary gland, adipose and prostate. ADAMTS-12 has been shown to be overproduced in gastrointestinal tumors and may be overexpressed in other cancer cells. Experiments have shown (see the Examples) that expression of ADAMTS-12 results in cells that are more invasive, e.g., more likely to be metastatic. Accordingly, assays can be performed using cancerous cells, e.g., biopsied cells, to determine the aggressiveness of the cancer. Invasiveness assays are common in the field and examples are presented below.

A number of in vitro systems have been developed to asses the invasiveness of tumor cells (for a review, see Terranova, V. P., et al. (1986) J. Natl. Cancer Inst. 77:311-316). Several of the assays utilize tissues which contain membranes such as bladder well, amnion, lens capsule and chorioallantoic membranes. More commonly the Matrigel® type assays is used to assay a cells invasiveness. For example, Matrigel has been affixed to a filter in a Boyden chamber and used to assess the ability of malignant cells to penetrate through the filter (Albini, A., et al. (1987) Cancer Res. 47:3239-45). Many improvements have been made on the original Matrigel method. For example, new dying technology has been used to quantitate the number of invasive cells eliminating the need to manually count cells through a microscope. MTT dye is cleaved by mitochondrial hydrolase generating a formazan dye that is quantifiable by spectrophotometry (Sasaki, C. et al. (1998) BioTechniques 24:1038-1043). Crystal violet dye has also been used to quantitating then number of invaded cells in Matrigel (Saito, K., et al. (1997) Biol. Pharm. Bull. 20:345-8).

Host cells can be natural cells which naturally contain the ADAMTS-12 gene and have been modified using the Random Activation of Gene Expression (RAGE) technology to over express ADAMTS-12 (for details on the RAGE technology see WO 00/49162 and WO 99/15650, incorporated herein by reference). The RAGE technology provides methods of expressing an endogenous gene at levels higher than normally found in the cell without having to clone the gene. RAGE is based on the introduction of a transcriptional activator in to a genome by non-homologous recombination. Host cells can be modified by the introduction of a transcriptional activator by homologous recombination as described in WO 94//12650, WO 95/31560, WO 96/29411, U.S. Pat. No. 5,733,761 and U.S. Pat. No. 6,270,985.

Host cells are also useful for conducting cell-based assays involving the ADAMTS-12 or ADAMTS-12 fragments. Thus, a recombinant host cell expressing a native ADAMTS-12 is useful to assay for compounds that stimulate or inhibit ADAMTS-12 function.

Host cells are also useful for identifying ADAMTS-12 mutants in which these functions are affected. If the mutants naturally occur and give rise to a pathology, host cells containing the mutations are useful to assay compounds that have a desired effect on the mutant ADAMTS-12 (for example, stimulating or inhibiting function) which may not be indicated by their effect on the native ADAMTS-12.

Recombinant host cells are also useful for expressing the chimeric polypeptides described herein to assess compounds that activate or suppress activation by means of a heterologous domain, segment, site, and the like, as disclosed herein.

Further, mutant ADAMTS-12 can be designed in which one or more of the various functions is engineered to be increased or decreased and used to augment or replace ADAMTS-12 proteins in an individual. Thus, host cells can provide a therapeutic benefit by replacing an aberrant ADAMTS-12 or providing an aberrant ADAMTS-12 that provides a therapeutic result. In one embodiment, the cells provide ADAMTS-12 that are abnormally active.

In another embodiment, the cells provide ADAMTS-12 that is abnormally inactive. This ADAMTS-12 can compete with endogenous ADAMTS-12 in the individual.

In a related embodiment, the cell of the invention can produce abnormally low levels of ADAMTS-12. This can be done, for example, by a method called RNA interference (RNAi). The best developed RNAi method is one that employs the siRNA technology developed by Tuschl, et al. The siRNA technique is a method of post transnational gene silencing that is initiated by double stranded RNA that is homologous to the sequence of the gene to be silenced. The siRNA methodology is described in Elbashir, S. M., et al. (2001) Nature 411:494-8 and Elbashir, S. M., et al. (2001) EMBO J. 3:6877-88. siRNAs have been used, for example, to silence genes in Xenopus embryos (Zhou, Y. et al (2002) Nucleic Acids Res. 30:1664-9) and to silence human tissue factor expression (Holen, T, et al (2002) Nucleic Acids Res. 30:1757-66).

In another embodiment, cells expressing ADAMTS-12 that cannot be activated are introduced into an individual in order to compete with endogenous ADAMTS-12. Homologously recombinant host cells can also be produced that allow the in situ alteration of endogenous ADAMTS-12 polynucleotide sequences in a host cell genome. The host cell includes, but is not limited to, a stable cell line, cell in vivo, or cloned microorganism. This technology is more fully described in WO 93/09222, WO 91/12650, WO 91/06667, U.S. Pat. No. 5,272,071, and U.S. Pat. No. 5,641,670. Briefly, specific polynucleotide sequences corresponding to the ADAMTS-12 polynucleotides or sequences proximal or distal to a ADAMTS-12 gene are allowed to integrate into a host cell genome by homologous recombination where expression of the gene can be affected. In one embodiment, regulatory sequences are introduced that either increase or decrease expression of an endogenous sequence. Accordingly, a ADAMTS-12 protein can be produced in a cell not normally producing it. Alternatively, increased expression of ADAMTS-12 protein can be effected in a cell normally producing the protein at a specific level. Further, expression can be decreased or eliminated by introducing a specific regulatory sequence. The regulatory sequence can be heterologous to the ADAMTS-12 protein sequence or can be a homologous sequence with a desired mutation that affects expression. Alternatively, the entire gene can be deleted. The regulatory sequence can be specific to the host cell or capable of functioning in more than one cell type. Still further, specific mutations can be introduced into any desired region of the gene to produce mutant ADAMTS-12 proteins. Such mutations could be introduced, for example, into the specific functional regions such as the cyclic nucleotide-binding site.

In one embodiment, the host cell can be a fertilized oocyte or embryonic stem cell that can be used to produce a transgenic animal containing the altered ADAMTS-12 gene. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous ADAMTS-12e gene is selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a ADAMTS-12 protein and identifying and evaluating modulators of ADAMTS-12 protein activity.

Other examples of transgenic animals include non-human primates, sheep, dogs, cows, goats, chickens, and amphibians.

In one embodiment, a host cell is a fertilized oocyte or an embryonic stem cell into which ADAMTS-12 polynucleotide sequences have been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the ADAMTS-12 nucleotide sequences can be introduced as a transgene into the genome of a non-human animal, such as a mouse.

Animal based models for studying cancer in vivo are well known in the art (reviewed in Animal Models of Cancer Predisposition Syndromes, Hiai, H. and Hino,

0. (eds.) 1999, Progress in Experimental Tumor Research, Vol. 35; Clarke, A. R. (2000) Carcinogenesis 21:435-41) and include, for example, carcinogen-induced tumors (Rithidech, K. et al. (1999) Mutat. Res. 428:33-39; Miller, M. L. et al. (2000) Environ. Mol. Mutagen. 35:319-327), injection and/or transplantation of tumor cells into an animal, as well as animals bearing mutations in growth regulatory genes, for example, oncogenes (e.g., ras) (Arbeit, J. M. et al. (1993) Am. J. Pathol. 142:1187-1197; Sinn, E. et al. (1987) Cell 49:465-475; Thorgeirsson, SS et al. (2000) Toxicol. Lett. 112-113:553-555) and tumor suppressor genes (e.g., p53) (Vooijs, M. et al. (1999) Oncogene 18:5293-5303; Clark A. R. (1995) Cancer Metast. Rev. 14:125-148; Kumar, T. R. et al. (1995) J Intern. Med 238:233-238; Donehower, L. A. et al. (1992) Nature 356215-221). Furthermore, experimental model systems are available for the study of, for example, ovarian cancer (Hamilton, T. C. el al. (1984) Semin. Oncol 11:285-298; Rahman, N. A. et al. (1998) Mol. Cell. Endocrinol 145:167-174; Beamer, W. G. et al. (1998) Toxicol. Pathol. 26:704-710), gastric cancer (Thompson, J. et al. (2000) Int. J. Cancer 86:863-869; Fodde, R. et al. (1999) Cytogenet. Cell Genet. 86:105-111), breast cancer (Li, M. et al. (2000) Oncogene 19:1010-1019; Green, J. E. et al. (2000) Oncogene 19:1020-1027), melanoma (Satyamoorthy, K. et al. (1999) Cancer Metast. Rev. 18:401-405); lung cancer (Malkinson, A. M. (2001) Lung Cancer 32(3):265-79; Zhao, B. et al. (2001) Exp. Lung Res. 26(8):567-79); colon cancer (Taketo, M. M. and Takaku (2000) Hum. Cell 13(3):85-95; Fodde, R. and Smits, R. (2001) Trends. Mol. Med. 7(8):369-73); and prostate cancer (Shirai, T. et al. (2000) Mutat. Res. 462:219-226; Bostwick, D. G. et al. (2000) Prostate 43:286-294).

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the ADAMTS-12 protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems, which allow for regulated expression of the transgene. One example of such a system is the cre/loxP recombinase system of bacteriophage PI. For a description of the cre/loxP recombinase system, see, e.g., Lakso et al. (1992) PNAS 89:6232-6236. Another example of a recombinase system is the FLP recombinase system of S. cerevisiae (O'Gorman et al. (1991) Science 251:1351-1355. If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385: 810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, it is useful to provide non-human transgenic animals to assay in vivo ADAMTS-12 function, including cAMP interaction, the effect of specific mutant ADAMTS-12 on ADAMTS-12 function and cAMP interaction, and the effect of chimeric ADAMTS-12. It is also possible to assess the effect of null mutations, that is mutations that substantially or completely eliminate one or more ADAMTS-12 functions.

In general, methods for producing transgenic animals include introducing a nucleic acid sequence according to the present invention, the nucleic acid sequence capable of expressing the protein in a transgenic animal, into a cell in culture or in vivo. When introduced in vivo, the nucleic acid is introduced into an intact organism such that one or more cell types and, accordingly, one or more tissue types, express the nucleic acid encoding the protein. Alternatively, the nucleic acid can be introduced into virtually all cells in an organism by transfecting a cell in culture, such as an embryonic stem cell, as described herein for the production of transgenic animals, and this cell can be used to produce an entire transgenic organism. As described, in a further embodiment, the host cell can be a fertilized oocyte. Such cells are then allowed to develop in a female foster animal to produce the transgenic organism.

V. Methods of Using ADAM-TS Modulators

Modulators of ADAMTS-12 level or activity identified according to these assays can be used to test the effects of modulation of expression of the enzyme on the outcome of clinically relevant disorders. This can be accomplished in vitro, in vivo, such as in human clinical trials, and in test models derived from other organisms, such as non-human transgenic subjects. Modulation in such subjects includes, but is not limited to, modulation of the cells, tissues, and disorders particularly disclosed herein. Modulators of ADAMTS-12 activity identified according to these drug screening assays can be used to treat a subject with a disorder mediated by ADAMTS-12, by treating cells that express the metalloprotease, such as those disclosed herein. In one embodiment, the cells that are treated are derived cancerous tissue or tumors. Accordingly, disorders in which modulation is particularly relevant can include these tissues. These methods of treatment include the steps of administering the modulators of ADAMTS-12 activity in a pharmaceutical composition as described herein, to a subject in need of such treatment.

The invention thus provides methods for treating a disorder characterized by aberrant expression or activity of a metalloprotease, e.g., ADAMTS-12. In one embodiment, the method involves administering an agent (e.g., an agent identified by a screening assay described herein), or combination of agents that modulates (e.g., upregulates or downregulates) expression or activity of the protein. In another embodiment, the method involves administering the metalloprotease as therapy to compensate for reduced or aberrant expression or activity of the protein.

Methods for treatment include but are not limited to the use of soluble ADAMTS-12 or fragments of ADAMTS-12 protein that compete for substrate. ADAMTS-12 or fragments thereof, can have a higher affinity for the target so as to provide effective competition.

Stimulation of activity is desirable in situations in which the protein is abnormally downregulated and/or in which increased activity is likely to have a beneficial effect. Likewise, inhibition of activity is desirable in situations in which the protein is abnormally upregulated and/or in which decreased activity is likely to have a beneficial effect. In one example of such a situation, a subject has a disorder characterized by aberrant development or cellular differentiation. In another example, the subject has a proliferative disease (e.g., cancer).

Pharmaceutical Compositions

The invention encompasses use of the polypeptides, nucleic acids, and other agents in pharmaceutical compositions to administer to the cells in which expression of the phosophodiesterase is relevant and in disorders as disclosed herein. Uses are both diagnostic and therapeutic. The ADAMTS-12 nucleic acid molecules, protein, modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier. It is understood however, that administration can also be to cells in vitro as well as to in vivo model systems such as non-human transgenic animals.

The term “administer” is used in its broadest sense and includes any method of introducing the compositions of the present invention into a subject. This includes producing polypeptides or polynucleotides in vivo as by transcription or translation, in vivo, of polynucleotides that have been exogenously introduced into a subject. Thus, polypeptides or nucleic acids produced in the subject from the exogenous compositions are encompassed in the term “administer.”

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a ADAMTS-12 protein or anti-ADAMTS-12 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tab lets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. The pharmaceutical compositions can be included in a container, pack, or dispenser together with instructions for administration.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Accordingly, the invention provides methods of treatment, with the nucleic acid as a target, using a compound identified through drug screening as a gene modulator to modulate ADAMTS-12 nucleic acid expression. Modulation includes both up-regulation (i.e. activation or agonization) or down-regulation (suppression or antagonization) or effects on nucleic acid activity (e.g. when nucleic acid is mutated or improperly modified). Disorders characterized by aberrant expression or activity of the nucleic acid can be treated.

The gene is particularly relevant for the treatment of disorders involving the cells and tissues that differentially express ADAMTS-12, cells that are involved in cell proliferative disorders and cells and tissues that are involved in metastasis.

Alternatively, a modulator for ADAMTS-12 nucleic acid expression can be a small molecule or drug identified using the screening assays described herein as long as the drug or small molecule inhibits the ADAMTS-12 nucleic acid expression.

The invention is further illustrated by the following examples, which should not be construed as further limiting. The contents of all references, pending patent applications and published patents, cited throughout this application are hereby expressly incorporated by reference. Those skilled in the art will understand that this invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### EXAMPLES

### Example I

### Expression of ADAMTS-12

ADAMTS-12 expression was analyzed by RT-PCR to determine the pattern of endogenous expression in selected human tissues, as follows: Gene specific (GS) forward and reverse PCR primers were designed and validated in control reactions using plasmid DNA purified from their corresponding cDNA clones. Primers were typically 24-28 nt in length, ≧50% G:C, and produced 200-300 bp amplification products. Control reactions were performed in 15 μl volumes containing 1×PCR buffer and 0.4 U Platinum Taq polymerase (LT1), 4 mM MgCl2, 0.23 mM dNTP's, 7.5 pmol of each GS primer, and 1 fg of plasmid template. After initial thermal denaturation at 95° C./2 min., reactions were performed by incubating at (95° C./15 sec.; 58° C./30 sec.; 72° C./40 sec) for ten cycles, (95° C./15 sec.; 63° C. 130 sec.; 72° C./40 sec.) for 25 cycles, and 72° C./2 minutes. Amplification products were resolved on 2% agarose gels containing CyberGold (Molecular Probes) and visualized on a transilluminator. GS primers failing to produce the expected amplification product were redesigned. Preparations of tissue specific (TS) total RNA's purified from normal human kidney, skeletal muscle, placenta, lung, spleen, liver, heart, adipose, prostate, mammary, pancreas, thymus, brain, cerebellum, salivary gland, uterus, colon, stomach, small intestine, adrenal gland, lymph node, thyroid, bladder and fetal liver and brain tissues were purchased from Clontech.

Total RNA was purified from cultured HT1080 cells using TriZol Reagent (LT1) following the manufacturer's recommended protocol. All RNA preparations were further treated to remove contaminating genomic by mixing 20 μg RNA with a premix of 0.5×Universal Buffer (Stratagene), 0.01 mM DTT, 40 U RNAsin (Promega), 20 U RNAse-free DNAse (Stratgene) to achieve a final volume of 100 μl. Reaction mixes were incubated at 37° C./30 minutes, then extracted once with an equal volume of phenol:chloroform (1:1). Samples were centrifuged, the aqueous phases transferred to fresh tubes and RNA's were precipitated by mixing 1× volume 3M sodium acetate and 2.5× volume absolute ethanol, incubating at −80° C./30 minutes and centrifuging. RNA pellets were rinsed with 80% ethanol, air-dried, and solvated in 100 μl dH20. cDNA reactions containing reverse transcriptase (+RT) and control reactions lacking reverse transcriptase (−RT) were performed by mixing 50 μl (approx. 10 μg) of DNAse-treated RNA with 50 μl of premixed 2×1 st Strand Buffer (LT1), 20 mM DTT, 1 mM dNTP's, 40 U RNAsin, 50 pmol primer GDR3V. (5′TCAGACTTAGATGGCCAACGAGGCCATTTTTTTT I-I-I-I TTTTT[A/C/G]3′) and either no added reverse transcriptase (−RT control reaction) or 300 U LT1 SuperScript II (+RT reaction). Mixtures were chilled to 16° C./6 minutes, incubated at 37° C./60 minutes, then incubated at 92° C./2 minutes. +RT and −RT reactions were validated for the absence of contaminating genomic DNA by PCR analysis using forward primer 159F12F (5′AACCCAGCGTTGGACAAATACIT3′) and reverse primer 159F12R (5′TAGCGCCATCATTCACATAATACAT3′) specific to exons 9 and 10, respectively, of human ornithine decarboxylase (ODC). PCR reaction chemistry and cycling conditions were the same as those described above. A single amplification product of 201 bp is expected from ODC cDNA, whereas an amplification product of 220 bp generated from the +RT cDNA reaction and/or −RT control reaction is diagnostic for the persistence of contaminating genomic DNA, and such cDNA samples were typically remade. Validated GS primers and a panel of 22 TS cDNA preparations were used in PCR analysis to determine expression profiles for each novel gene by adding 0.5 μg of cDNA into a premix containing 1×PCR buffer, 0.23 mM dNTP's, 4 mM MgCl2, 7.5 pmol each of forward and reverse GS primer, and 0.4 U Platinum Taq polymerase at a final reaction volume of 15 μl. Conditions for PCR cycling and the detection of amplification products were the same as those described above.

Two independent RT-PCR experiments using different primer pairs generated products corresponding to the size predicted for ADAMTS-12 cDNA amplification were reproducibly seen in kidney, skeletal muscle, spleen, fetal liver, heart, stomach, uterus, salivary gland, adipose and prostate tissues.

### Example II

### Invasiveness of Cells Transfected with ADAMTS-12

The following example describes experiments that demonstrate the expression of ADAMTS-12 increases the invasiveness of cells. Using a Matrigel migration assay it is shown that cells that do not have the ability to migrate through the artificial basement membrane Matrigel gain that ability when transfected with a plasmid expressing ADAMTS-12.

Cloning:

A full-length clone of ADAM-TS 12 (GenBank accession no. AJ250725) was generated by PCR and inserted into the vector pcDNA3.1 with a FLAG epitope tag added to the carboxy-terminus.

Cell Line:

H4 neuroglioma cells were obtained from ATCC (HTB-148) and cultured in DMEM containing 10% FBS, 2 mM glutamine, and 1 μM each of penicillin and streptomycin.

Matrigel Migration Assay:

H4 cells (10,000) containing pcDNA3.1 with ADAM-TS 12, or pcDNA3.1 only, were seeded in 100 μl volume onto 24-well 8-μm inserts (BD labware) coated with Matrigel (BD labware). The inserts. were placed into a 24-well plate containing 600 μl DMEM with 5% FBS. The cells were incubated for 24 hrs in a 38° C. incubator with 5% CO₂. The cells on the top of the insert were scraped off, and cells that had migrated across the insert were stained with Diff-Quik (Sigma) and the number of cells in five fields counted by bright-field microscopy using a 10× objective. All studies were performed in triplicate.

Zymogram:

H4 cells (10,000) containing pcDNA3.1 with ADAMTS-12, or pcDNA3.1 only, were incubated for 24 hrs in serum-free DMEM. The media was collected and the cells lysed in 1% Triton X-100, 150 mM NaCl and 50 mM Tris-HCl, pH 8.0 for 30 min on ice. The media and the cell lysates were then clarified by centrifugation for 10 min at 14,000×g. A total of 20 μg of the cell lysate or 20 μl of the media were mixed with non-reducing gel loading buffer and loaded onto a 10% SDS-PAGE gel containing 1 mg/ml gelatin. The gel was run under non-reducing conditions at 100V at 4° C. The gel was then placed in 2.5% Triton X-100 for 30 min to remove SDS and allow the proteases to renature, and was then incubated for 3 days in 100 mM Tris-CI, pH 8.0 and 5 mM CaCl2. The gel was stained with Coomassie blue and destained to visualize degraded gelatin.

Western Blot Analysis:

Cell lysates and media were prepared as described above and analyzed by SDS-PAGE electrophoresis under reducing conditions. The gels were then transferred to nitrocellulose, blocked with 5% non-fat dry milk in TBST and incubated with a monoclonal antibody against the FLAG epitope (Sigma) for 1 hr at rt. After 3 washes, the nitrocellulose blot was incubated for 30 min with an HRP-conjugated anti-mouse antibody, washed and FLAG immunoreactivity was detected by chemiluminescence (ECL kit).

Results and Discussion

H4 cells were first transiently transfected with ADAM-TS12 containing the FLAG epitope at the carboxy-terminus, or with vector alone. The expression of ADAM-TS12

was confirmed by Western blot and full-length ADAM-TS12 was detected. A similar result was obtained in cells that were stably transfected with ADAM-TS 12. This is in contrast to a recent published study looking at ADAM-TS 12 expression in COS-7 cells (Gal et al. (2001) J. Biol. Chem. 276:17932), where an 83 kD COOH-terminal cleavage product was also detected. It is possible that the post-translation processing in COS-7 cells is different than that of H4 cells, that the epitope tag is cleaved during processing, or that the levels of the 83 kD product in H4 cells were below detection.

Numerous studies (Albini et al. (1987) Cancer Res. 47:3239, Parish et al., (1992) Int. J. Cancer 52:378, Sato et al. (1994) Nature 370:61, for review see Albini (1998) Pathol Oncol Res. 4:230) have demonstrated that cancer cells with high metastatic potential can migrate through the artificial basement membrane, Matrigel, whereas non-metastatic cells do not traverse Matrigel. A Matrigel assay was used to examine the invasiveness of H4 cells expressing ADAM-TS12. Cells that expressed ADAM-TS12 had a greater than 2-fold increase in the number of invasive cells when compared to cells transfected with vector only. These results are similar to what was observed in H4 cells expressing uPAR (Mohanam et al. (1998) Int. J. Oncol. 13:1285). In those studies, the expression of uPAR increased the invasiveness of H4 cells by almost 2-fold. This enzyme is involved in the activation of plasminogen to plasmin, with subsequent degradation of extracellular matrix. There are a number of studies that implicate increased uPAR activity with tumor cell invasion and metastasis. Thus, the fact that ADAM-TS 12 expression gave a similar increase in the level of invasive activity suggests that it may also play a role in tumor cell metastasis. Additionally, while the endogenous expression of ADAM-TS 12 is primarily in fetal lung, certain tumors, including gastric tumors, over-express ADAM-TS 12 (Cal et al. (2001) J. Biol. Chem. 276:17932) further suggesting a role in cancer.

In order to determine whether there was any novel proteolytic activity associated with ADAM-TS 12 expression, a zymogram was performed using gelatin as a substrate. The H4 cells have an ˜90 kD activity that was associated only with the ADAM-TS 12 transfected cells. Furthermore, this activity was significantly more pronounced in the media than the cell lysates. The simplest interpretation of these results is that the gelatinolytic activity is due directly to ADAM-TS 12 itself.

1. A method for identifying an agent that modulates the level or activity of a polypeptide in a cell, wherein said polypeptide is selected from the group consisting of:
(a) The amino acid sequence shown in SEQ ID NO:1;
(b) The amino acid sequence of an allelic variant of the amino acid sequence shown in SEQ ID NO: 1;
(c) The amino acid sequence of a sequence variant of the amino acid sequence shown in SEQ ID NO: 1, wherein the sequence variant is encoded by a nucleic acid molecule hybridizing to the nucleic acid molecule shown in SEQ ID NO:2 under stringent conditions;
(d) A fragment of the amino acid sequence shown in SEQ ID NO. 1, wherein the fragment comprises at least 10 contiguous amino acids;
(e) The amino acid sequence of an epitope bearing region of anyone of the polypeptides of (a)-(d);
said method comprising: contacting said agent with a cell capable of expressing said polypeptide such that said polypeptide level or activity can be modulated in said cell by said agent and measuring said polypeptide level or activity.

2. A method of screening a cell to identify an agent that modulates the level or activity of a polypeptide in said cell, wherein said polypeptide is selected from the group consisting of:
(a) The amino acid sequence shown in SEQ ID NO: 1;
(b) The amino acid sequence of an allelic variant of the amino acid sequence shown in SEQ ID NO: 1;
(c) The amino acid sequence of a sequence variant of the amino acid sequence shown in SEQ ID NO: 1, wherein the sequence variant is encoded by a nucleic acid molecule hybridizing to the nucleic acid molecule shown in SEQ ID NO:2 under stringent conditions;
(d) A fragment of the amino acid sequence shown in SEQ ID NO: 1, wherein the fragment comprises at least 10 contiguous amino acids;
(e) The amino acid sequence of an epitope bearing region of anyone of the polypeptides of (a)-(d);
said method comprising: contacting said agent with a cell capable of expressing said polypeptide such that said polypeptide level or activity can be modulated in said cell by said agent and measuring said polypeptide level or activity.

3. The method of claim 1 wherein said cell is derived from a subject with cancer.

4. The method of claim 3 wherein said cell is derived from a subject with metastatic cancer.

5. The method of claim 1 wherein said agent decreases the level or activity of said polypeptide.

6. A method for identifying an agent that interacts with a polypeptide in a cell, wherein said polypeptide is selected from the group consisting of:
(a) The amino acid sequence shown in SEQ ID NO: 1;
(b) The amino acid sequence of an allelic variant of the amino acid sequence shown in SEQ ID NO:1;
(c) The amino acid sequence of a sequence variant of the amino acid 15 sequence shown in SEQ ID NO: 1, wherein the sequence variant is encoded by a nucleic acid molecule hybridizing to the nucleic acid molecule shown in SEQ ID NO:2, under stringent conditions;
(d) A fragment of the amino acid sequence shown in SEQ ID NO: 1, wherein the fragment comprises at least 10 contiguous amino acids;
(e) The amino acid sequence of an epitope bearing region of anyone of the polypeptides of (a)-(d);
said method comprising: contacting said agent with a cell capable of allowing an interaction between said polypeptide and said agent such that said polypeptide can interact with said agent and measuring the interaction.

7. A method of screening a cell to identify an agent that interacts with a polypeptide in a cell, wherein said polypeptide is selected from the group consisting of:
(a) The amino acid sequence shown in SEQ ID NO: 1;
(b) The amino acid sequence of an allelic variant of the amino acid sequence shown in SEQ ID NO: 1;
(c) The amino acid sequence of a sequence variant of the amino acid sequence shown in SEQ ID NO: 1, wherein the sequence variant is encoded by a nucleic acid molecule hybridizing to the nucleic acid molecule shown in SEQ ID NO:2, respectively, under stringent conditions;
(d) A fragment of the amino acid sequence shown in SEQ ID NO: 1, wherein the fragment comprises at least 10 contiguous amino acids;
(e) The amino acid sequence of an epitope bearing region of any one of the polypeptides of (a)-(d);
said method comprising: contacting said agent with a cell capable of allowing an interaction between said polypeptide and said agent such that said polypeptide can interact with said agent and measuring the interaction.

8. The method of claim 6, said method comprising:
(1) exposing said agent to said polypeptide under conditions that allow said agent to interact with said polypeptide; (2) adding competing polypeptide that can interact with said agent; and (3) comparing the amount of interaction between said agent and said polypeptide to the amount of interaction in the absence of said competing polypeptide.

9. The method of claim 6 wherein said interaction is binding.

10. The method of claim 1 wherein said agent increases interaction between said polypeptide and a target molecule for said polypeptide, said method comprising: combining said polypeptide with said agent under conditions that allow said polypeptide to interact with said target molecule; and detecting the formation of a complex between said polypeptide and said target molecule or activity of said polypeptide as a result of interaction of said polypeptide with said target molecule.

11. The method of claim 1 wherein said agent decreases interaction between said polypeptide and a target molecule for said polypeptide, said method comprising: combining said polypeptide with said agent under conditions that allow said polypeptide to interact with said target molecule; and detecting the formation of a complex between said polypeptide and said target molecule or activity of said polypeptide as a result of interaction of said polypeptide with said target molecule.

12. The method of claim 1 wherein said cell is in vivo.

13. The method of claim 12 wherein said cell is in a transgenic animal.

14. The method of claim 12 wherein said cell is in a non-transgenic subject.

15. The method of claim 1 wherein said cell is in vitro.

16. The method of claim 15 wherein said cell has been disrupted.

17. The method of claim 15 wherein said cell is in a biopsy.

18. The method of claim 16 wherein said cell is in cell culture.

19. The method of claim 18 wherein said cell is naturally-occurring or recombinant.

20. The method of claim 1 wherein said agent is selected from the group consisting of a peptide; antibody; organic molecule; and inorganic molecule.

21. A method for detecting the presence of a polypeptide in a sample, said method comprising contacting said sample with an agent that specifically allows detection of the presence of the polypeptide in the sample and then detecting the presence of the polypeptide, wherein said polypeptide is selected from the group consisting of:
(a) The amino acid sequence shown in SEQ ID NO: 1;
(b) The amino acid sequence of an allelic variant of the amino acid sequence shown in SEQ ID NO: 1;
(c) The amino acid sequence of a sequence variant of the amino acid sequence shown in SEQ ID NO: 1, wherein the sequence variant is encoded by a nucleic acid molecule hybridizing to the nucleic acid molecule shown in SEQ ID NO:2 under stringent conditions;
(d) A fragment of the amino acid sequence shown in SEQ ID NO: 1, wherein the fragment comprises at least 10 contiguous amino acids;
(e) The amino acid sequence of an epitope bearing region of anyone of the polypeptides of (a)-(e);
wherein said sample is derived from cells involved in abnormal cell proliferation.

22. The method of claim 21, wherein said agent is capable of selective physical association with said polypeptide.

23. The method of claim 22, wherein said agent binds to said polypeptide.

24. The method of claim 23, wherein said agent is an antibody.

25. A kit comprising reagents used for the method of claim 21, wherein the reagents comprise an agent that specifically binds to said polypeptide.

26. A method for modulating the level or activity of a polypeptide, the method comprising contacting said polypeptide with an agent under conditions that allow the agent to modulate the level or activity of the polypeptide, wherein said polypeptide is selected from the group consisting of:
(a) The amino acid sequence shown in SEQ ID NO: 1;
(b) The amino acid sequence of an allelic variant of the amino acid sequence shown in SEQ ID NO: 1;
(c) The amino acid sequence of a sequence variant of the amino acid sequence shown in SEQ ID NO: 1, wherein the sequence variant is encoded by a nucleic acid molecule hybridizing to the nucleic acid molecule shown in SEQ ID NO: 2 under stringent conditions;
(d) A fragment of the amino acid sequence shown in SEQ ID NO: 1, wherein the fragment comprises at least 10 contiguous amino acids;
(e) The amino acid sequence of an epitope bearing region of anyone of the polypeptides of (a)-(d);
wherein said modulation occurs in a cancer cell.

27. A method for identifying an agent that modulates the level or activity of a nucleic acid molecule in a cell, wherein said nucleic acid molecule has a nucleic acid sequence selected from the group consisting of:
(a) The nucleotide sequence shown in SEQ ID NO:2;
(b) A nucleotide sequence encoding the amino acid sequence shown in SEQ ID NO: 1;
(c) A nucleotide sequence complementary to any of the nucleotide sequences in (a), (b), or (c).
(d) A nucleotide sequence encoding an amino acid sequence of a sequence variant of the amino acid sequence shown in SEQ ID NO: 1 that hybridizes to the nucleotide sequence shown in SEQ ID NO:2 under stringent conditions;
(e) A nucleotide sequence encoding a fragment of the amino acid sequence shown in SEQ ID NO: 1, wherein the fragment comprises at least 10 contiguous amino acids;
said method comprising contacting said agent with a cell capable of expressing said nucleic acid molecule such that said nucleic acid molecule level or activity can be modulated in said cell by said agent and measuring said nucleic acid molecule level or activity.

28. A method of screening a cell to identify an agent that modulates the level or activity of a nucleic acid molecule in said cell, wherein said nucleic acid molecule has a nucleotide sequence selected from the group consisting of:
(a) The nucleotide sequence shown in SEQ ID NO:2;
(b) A nucleotide sequence encoding the amino acid sequence shown in SEQ ID NO: 1;
(c) A nucleotide sequence complementary to any of the nucleotide sequences in (a) or (b);
(d) A nucleotide sequence encoding an amino acid sequence of a sequence variant of the amino acid sequence shown in SEQ ID NO: 1 that hybridizes to the nucleotide sequence shown in SEQ ID NO:2 under stringent conditions;
(e) A nucleotide sequence encoding a fragment of the amino acid sequence shown in SEQ ID NO: 1, wherein the fragment comprises at least 10 contiguous amino acids;
said method comprising: contacting said agent with a cell capable of expressing said nucleic acid molecule such that said nucleic acid molecule level or activity can be modulated in said cell by said agent and measuring nucleic acid molecule level or activity.

29. A method for identifying an agent that interacts with a nucleic acid molecule in a cell, wherein said nucleic acid molecule has a nucleotide sequence selected from the group consisting of:
(a) The nucleotide sequence shown in SEQ ID NO:2;
(b) A nucleotide sequence encoding the amino acid sequence shown in SEQ ID NO: 1;
(c) A nucleotide sequence complementary to any of the nucleotide sequences in
(a) or (b)
(d) A nucleotide sequence encoding an amino acid sequence of a sequence variant of the amino acid sequence shown in SEQ ID NO: 1 that hybridizes to the nucleotide sequence shown in SEQ ID No12 under stringent conditions;
(e) A nucleotide sequence encoding a fragment of the amino acid sequence shown in SEQ ID NO: 1, wherein the fragment comprises at least 10 contiguous amino acids;
said method comprising: contacting said agent with a cell capable of allowing an interaction between said nucleic acid molecule and said agent, such that nucleic acid molecule can interact with said agent and measuring the interaction.

30. A method of screening a cell to identify an agent that interacts with a nucleic acid molecule in a cell, wherein said nucleic acid molecule has a nucleotide sequence selected from the group consisting of:
(a) The nucleotide sequence shown in SEQ ID NO: 2;
(b) A nucleotide sequence encoding the amino acid sequence shown in SEQ ID NO: 1;
(c) A nucleotide sequence complementary to any of the nucleotide sequences in
(a) or (b);
(d) A nucleotide sequence encoding an amino acid sequence of a sequence variant of the amino acid sequence shown in SEQ ID NO: 1 that hybridizes to the nucleotide sequence shown in SEQ ID NO:2 under stringent conditions;
(e) A nucleotide sequence encoding a fragment of the amino acid sequence shown in SEQ ID NO: 1, wherein the fragment comprises at least 10 contiguous amino acids;
said method comprising: contacting said agent with a cell capable of allowing an interaction between said nucleic acid molecule and said agent, such that nucleic acid molecule can interact with said agent and measuring the interaction.

31. A method for detecting the presence of a nucleic acid molecule in a sample, said method comprising contacting said sample with an agent that specifically allows detection of the presence of the nucleic acid molecule in the sample and then detecting the presence of the nucleic acid molecule, the nucleic acid molecule having a nucleotide sequence selected from the group consisting of:
(a) The nucleotide sequence shown in SEQ ID NO: 2;
(b) A nucleotide sequence encoding the amino acid sequence shown in SEQ ID NO: 1;
(e) A nucleotide sequence complementary to any of the nucleotide sequences in (a) or (b)
(d) A nucleotide sequence encoding an amino acid sequence of a sequence variant of the amino acid sequence shown in SEQ ID NO: 1 that hybridizes to the nucleotide sequence shown in SEQ ID NO: 2 under stringent conditions;
(e) A nucleotide sequence encoding a fragment of the amino acid sequence shown in SEQ ID NO: 1, wherein the fragment comprises at least 10 contiguous amino acids;
wherein said sample is derived from cells involved in abnormal cell proliferation.

32. The method of claim 31, wherein the method comprises contacting the sample with an oligonucleotide that hybridizes to the nucleic acid sequences of (a)-(e) under stringent conditions and determining whether the oligonucleotide binds to the nucleic acid sequence in the sample.

33. The method of claim 32, wherein the nucleic acid whose presence is detected is mRNA.

34. A kit comprising reagents used for the method of claim 32, wherein the reagents comprise a compound that hybridizes under stringent conditions to any of the nucleic acid molecules.

35. The method of claim 34 wherein a fragment of the nucleic acid is contacted.

36. A method for modulating the level or activity of a nucleic acid molecule, said method comprising contacting said nucleic acid molecule with an agent under conditions that allow the agent to modulate the level or activity of the nucleic acid molecule, said nucleic acid molecule having a nucleotide sequence selected from the group consisting of:
(a) The nucleotide sequence shown in SEQ ID NO: 2;
(b) A nucleotide sequence encoding the amino acid sequence shown in SEQ ID NO: 1;
(c) A nucleotide sequence complementary to any of the nucleotide sequences in (a), (b)
(d) A nucleotide sequence encoding an amino acid sequence of a sequence variant of the amino acid sequence shown in SEQ ID NO: 1 that hybridizes to the nucleotide sequence shown in SEQ ID NO: 2 under stringent conditions;
(e) A nucleotide sequence encoding a fragment of the amino acid sequence shown in SEQ ID NO: 1, wherein the fragment comprises at least 10 contiguous amino acids;
wherein said modulation is in a cancer cell.

37. The method of claim 21 wherein said detecting is in a cell derived from a subject having a disorder or predisposed to having a disorder involving said cell.

38. The method of claim 26 wherein said modulation is in a cell derived from a subject having a disorder or predisposed to having a disorder involving said cell.

39. The method of claim 37 wherein said disorder is selected from the group consisting of cancer, inflammation or cardiovascular disease.

40. The method of claim 38 wherein said disorder is selected from the group consisting of cancer, inflammation or cardiovascular disease.

41. The method of claim 38 wherein said cell is in a nonhuman transgenic animal.

42. An agent identified by any of the methods of claims 1, 2, 6 or 7.

43. A method of treating an individual having an ADAMTS-12 related disorder said method comprising:
administering to said individual an effective amount of a ADAMTS inhibitor
such that ADAMTS activity is inhibited in said individual.

44. A method for modulating extracellular matrix degradation by controlling the level or activity of ADAMTS-12 or a portion thereof.

45. The method of claim 44, wherein the modulation of extracellular matrix degradation is in vivo.

46. The method of claim 44, wherein the modulation of extracellular matrix degradation is in vitro.

47. The method of claim 44, wherein the extracellular matrix is associated with a tumor.

48. The method of claim 44, wherein the extracellular matrix is associated with metastasis.

49. The method of claim 44, wherein the portion of ADAMTS-12 is a domain of ADAMTS-12 polypeptide.

50. The method of claim 49, wherein the domain is selected from the group consisting of a catalytic domain, prodomain, thrombospondin domain, a cysteine rich domain and a reprolyrin domain.

51. The method of claim 44, wherein the degradation is of an artificial basement membrane.

52. The method of claim 44, wherein the modulation causes a decrease in breakdown of extracellular matrix.

53. The method of claim 44, wherein the modulation decreases or prevents breakdown of extracellular matrix.
